SEQUENCE LISTING (1 sequence)

SEQ ID NO: 1 - DNA, 113 bp, synthetic construct
  source 1..113: mol_type = other DNA
ctcacgccttatgactgcccttatgtcaccgcttatgtctcccgatatcacacccgttatctcagccctaatctctgcggtttagtctggccttaatccatgcctcatagcta

(19) United States
(12) Patent Application Publication
(10) Pub. No.: US 2025/0290921 A1
(43) Pub. Date: Sep. 18, 2025

(54) PARTICLE ENCAPSULATION METHOD, MICROWELL DEVICE, AND TARGET SUBSTANCE DETECTION METHOD

(71) Applicant: CANON KABUSHIKI KAISHA, Tokyo (JP)

(72) Inventors: SHIGEMOTO ABE, Kanagawa (JP); DAISUKE SASAGURI, Kanagawa (JP); SATOSHI OGAWA, Kanagawa (JP); KYOKO BABA, Kanagawa (JP)

(21) Appl. No.: 19/076,384

(22) Filed: Mar. 11, 2025

(30) Foreign Application Priority Data
Mar. 18, 2024 (JP) 2024-042796
Feb. 6, 2025 (JP) 2025-018577

(51) Int. Cl. G01N 33/543; B01L 3/00

(52) U.S. Cl. CPC G01N 33/54313; B01L 3/50853; G01N 33/54386

(57) ABSTRACT

Provided is a particle encapsulation method including: a preparing step of preparing a microwell device including: a base plate having arranged in an upper surface thereof an array formed of a plurality of microwells; and a wiping member; an introducing step of introducing a dispersion medium containing detection particles into the array; a packing step of packing the detection particles into the microwells; a removing step of removing the dispersion medium present outside the microwells; and a sealing step of sealing the microwells with a sealing medium, wherein the removing step includes sliding the wiping member in a first direction under a state in which the wiping member is brought into contact with the upper surface of the base plate, to thereby move the dispersion medium present outside the microwells to an outside of the array.

### BACKGROUND OF THE INVENTION

### Field of the Invention

The present invention relates to a particle encapsulation method, a microwell device, and a target substance detection method.

### Description of the Related Art

An approach called single-molecule measurement has been known as a method of performing various kinds of measurement by observing a biomolecule such as a protein after bringing the biomolecule into a distinguishable form, and some approaches for performing the single-molecule measurement have been known.

In International Publication No. WO2012/121310, there is a disclosure of a technology of encapsulating particles in microwells through use of the following configuration: a partition wall and a top plate are arranged on a microwell array to form a flow path; and an injection port and a blowout port are further arranged in the top plate. In International Publication No. WO2012/121310, when the particles are loaded into the microwells, a hydrophilic liquid medium containing the particles that have each captured a target substance is introduced from the injection port with the flow path. In addition, when the microwells are sealed, a hydrophobic liquid medium is similarly introduced from the injection port with the flow path to push out the redundant hydrophilic liquid medium that has become unnecessary.

In addition, in H. Yaginuma et al., Lab on a Chip, 2022, 22, 2001-2010, there is a disclosure of a technology of encapsulating particles in microwells by rolling a roller from above an adhesive tape, which is arranged so as to cover the microwells, a plurality of times while pressing the roller against the tape. In H. Yaginuma et al., Lab on a Chip, 2022, 22, 2001-2010, a hydrophilic liquid medium containing the particles is directly introduced into each of the microwells by arranging partition walls on a microwell array with a Kapton tape. After that, the adhesive tape is arranged so as to bridge the partition walls, and the roller is rolled from above the tape a plurality of times while being pressed against the tape. Thus, the redundant hydrophilic liquid medium that has become unnecessary is pushed out, and hence the microwells are sealed.

Each of the technologies described in International Publication No. WO2012/121310 and H. Yaginuma et al., Lab on a Chip, 2022, 22, 2001-2010 has involved a problem in that it is difficult to uniformly encapsulate the particles in the microwells.

### SUMMARY OF THE INVENTION

Accordingly, an object of the present invention is to provide a simple particle encapsulation method by which particles can be uniformly encapsulated in microwells.

To solve the above-mentioned problem, according to one aspect of the present invention, there is provided a particle encapsulation method including: a preparing step of preparing a microwell device including: a base plate having arranged in an upper surface thereof an array formed of a plurality of microwells; and a wiping member; an introducing step of introducing a dispersion medium containing detection particles into the array; a packing step of packing the detection particles into the microwells; a removing step of removing the dispersion medium present outside the microwells; and a sealing step of sealing the microwells with a sealing medium, wherein the removing step includes sliding the wiping member in a first direction under a state in which the wiping member is brought into contact with the upper surface of the base plate, to thereby move the dispersion medium to an outside of the array.

In addition, according to another aspect of the present invention, there is provided a microwell device including: a base plate having arranged in an upper surface thereof an array formed of a plurality of microwells; and a wiping member, wherein the microwells are wells for storing detection particles dispersed in a dispersion medium, and wherein the wiping member is configured to be capable of moving the dispersion medium present outside the microwells to an outside of the array by being slid in a first direction under a state of being brought into contact with the upper surface of the base plate.

In addition, according to another aspect of the present invention, there is provided a target substance detection method including: a reaction step of causing the detection particles that each specifically capture a target substance and the target substance to react with each other; a particle-encapsulating step of performing the above-mentioned particle encapsulation method according to one aspect of the present invention with the detection particles after the reaction step; and a detecting step of detecting the detection particles that have each captured the target substance out of the detection particles stored in the respective microwells after the particle-encapsulating step.

Further features of the present invention will become apparent from the following description of exemplary embodiments with reference to the attached drawings.

### BRIEF DESCRIPTION OF THE DRAWINGS

FIG. 1 is an exploded perspective view for illustrating an example of the configuration of a microwell device.

FIG. 2 is a perspective view for illustrating an example of the configuration of the microwell device.

FIG. 3A, FIG. 3B, FIG. 3C, FIG. 3D, FIG. 3E, FIG. 3F, and FIG. 3G are schematic views for describing the respective steps of a particle encapsulation method.

FIG. 4 is an exploded perspective view for illustrating an example of the configuration of the microwell device.

FIG. 5 is a perspective view for illustrating an example of the configuration of the microwell device.

FIG. 6A, FIG. 6B, FIG. 6C, FIG. 6D, FIG. 6E, FIG. 6F, and FIG. 6G are schematic views for describing the respective steps of the particle encapsulation method.

FIG. 7 is a perspective view for illustrating an example of the configuration of the microwell device.

FIG. 8 is a perspective view for illustrating an example of the configuration of the microwell device.

FIG. 9 is a perspective view for illustrating an example of the configuration of the microwell device.

### DESCRIPTION OF THE EMBODIMENTS

The present invention is described in detail below by way of exemplary embodiments.

In the technology of International Publication No. WO2012/121310, to uniformly pack the particles into the microwells, the rate at which the hydrophilic liquid medium containing the particles is injected needs to be unerringly controlled. In addition, when the hydrophobic liquid medium is injected, the hydrophobic liquid medium is continuously introduced from the injection port, and hence air bubbles are mixed in the hydrophobic liquid medium in some cases. Further, there has been a problem in that it is difficult to uniformly push out the entirety of the hydrophilic liquid medium to uniformly seal the microwells. Particularly when the microwell array has a large area, it becomes more difficult to uniformly push away a liquid to a wide flow path.

In addition, in the technology described in H. Yaginuma et al., Lab on a Chip, 2022, 22, 2001-2010, a technology of the application of a so-called window film to be performed by a professional craftsman needs to be applied, and hence it is difficult to uniformly seal the microwells so that no air bubbles may remain in the adhesive tape. Further, the technology described in the literature does not include any step of removing the particles that have not entered the microwells, and hence the microwells are sealed with the adhesive tape under a state in which the particles that have not entered the microwells stay. Accordingly, it may become difficult to secure an effective observation range for detection.

In other words, to appropriately perform each of the technologies described in International Publication No. WO2012/121310 and H. Yaginuma et al., Lab on a Chip, 2022, 22, 2001-2010, reasonable technique is required. When the technique is insufficient, unevenness occurs in the particles to be encapsulated in the microwells, or the number of the microwells having encapsulated therein the particles reduces in some cases. Alternatively, air bubbles are mixed in the liquid medium containing the particles at the time of the sealing of the microwells in some cases. As a result, the number of signals reduces or noise enlarges at the time of measurement, and hence the measurement finally requires a long time period in some cases. In addition, the redundant hydrophilic liquid medium that has been pushed out may stain an inspection apparatus including a microwell device, and the staining leads to labor for maintenance by a user.

The inventors of the present application have made extensive investigations with a view to solving the problems in the related art, and as a result, have achieved a particle encapsulation method according to the present invention to be described in detail below.

The particle encapsulation method according to the present invention includes a preparing step, an introducing step, a packing step, a removing step, and a sealing step. The preparing step is a step of preparing a microwell device including: a base plate having arranged in its upper surface an array formed of a plurality of microwells; and a wiping member. In addition, the introducing step is a step of introducing a dispersion medium containing detection particles into the above-mentioned array. In addition, the packing step is a step of packing the above-mentioned detection particles into the above-mentioned microwells. In addition, the removing step is a step of removing the above-mentioned dispersion medium present outside the above-mentioned microwells. In addition, the sealing step is a step of sealing the above-mentioned microwells with a sealing medium. Further, the above-mentioned removing step includes sliding the above-mentioned wiping member in a first direction under a state in which the member is brought into contact with the upper surface of the above-mentioned base plate, to thereby move the dispersion medium to the outside of the array.

The particle encapsulation method according to the present invention can shorten a time period up to measurement, enlarge a signal at the time of the measurement, and reduce a possibility that an inspection apparatus is stained.

The respective configurations of the present invention are described in detail below by way of exemplary embodiments of the present invention with reference to the drawings. Like elements or corresponding elements are denoted by the same reference numerals in the drawings, and description thereof may be omitted or simplified.

### <Microwell Device>

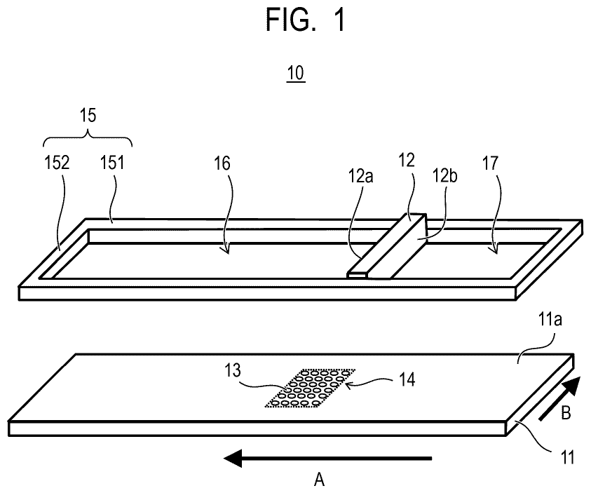

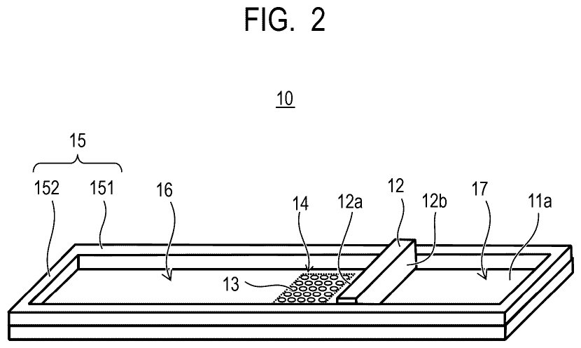

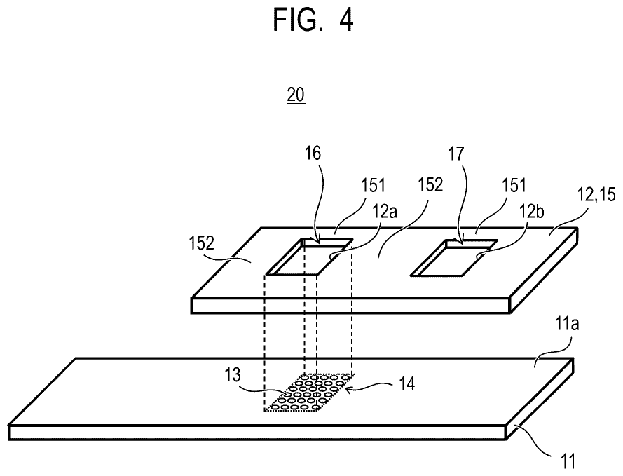

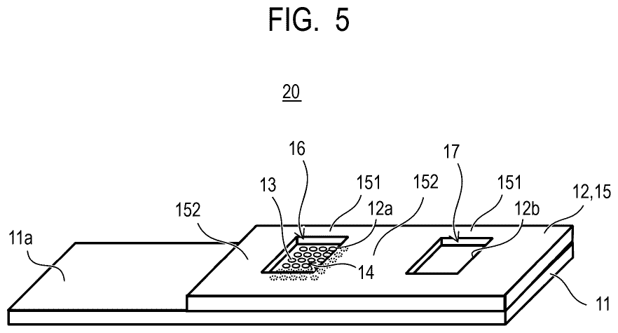

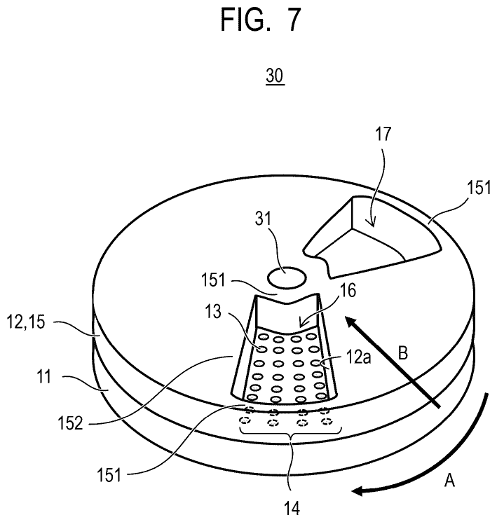

First, specific examples of the configuration of the microwell device that may be used in the particle encapsulation method according to the present invention are illustrated in FIG. 1, FIG. 2, FIG. 4, FIG. 5, and FIG. 7 to FIG. 9. FIG. 2, FIG. 5, and FIG. 7 to FIG. 9 are perspective views for illustrating the overall configurations of microwell devices 10 to 50. In addition, FIG. 1 is an exploded perspective view of the microwell device 10 illustrated in FIG. 2, and FIG. 4 is an exploded perspective view of the microwell device 20 illustrated in FIG. 5.

The microwell device according to the present invention includes: a base plate 11 having formed in an upper surface 11a thereof an array 14 formed of a plurality of microwells 13; and a wiping member 12.

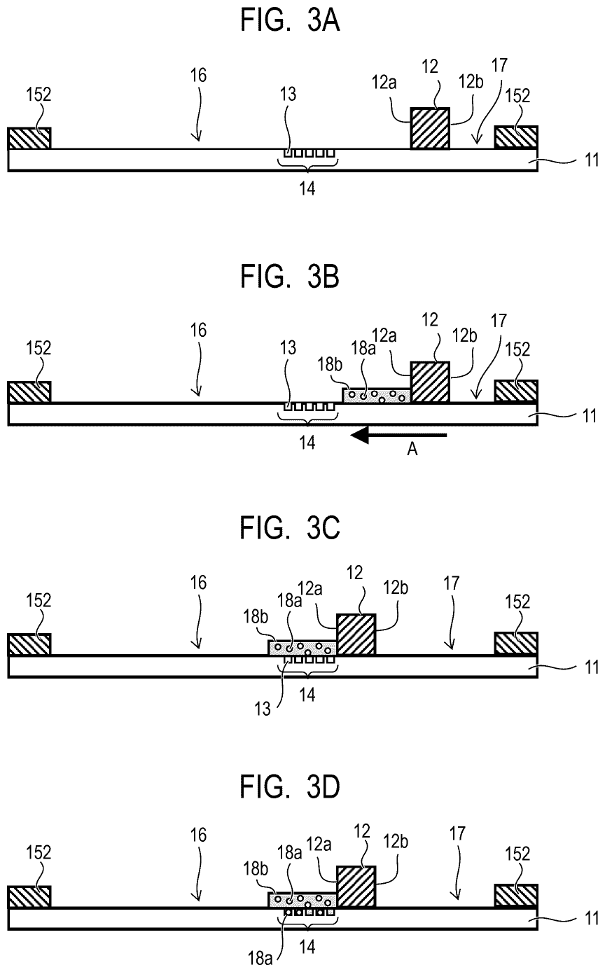

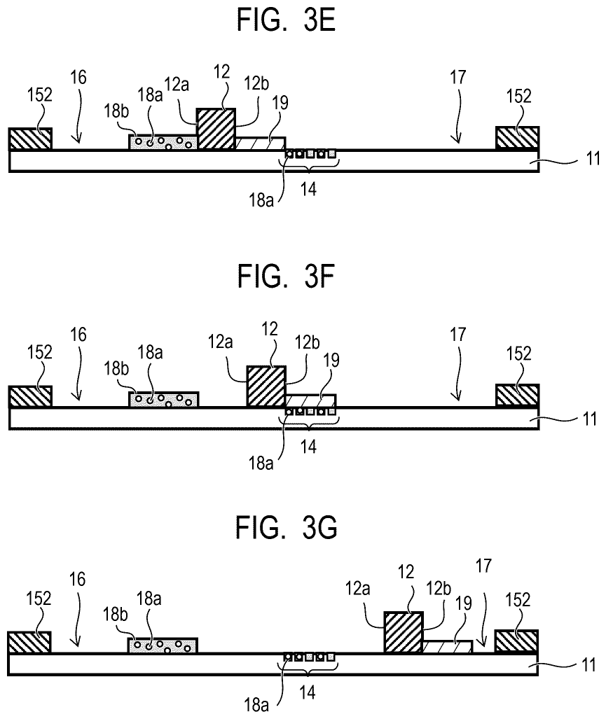

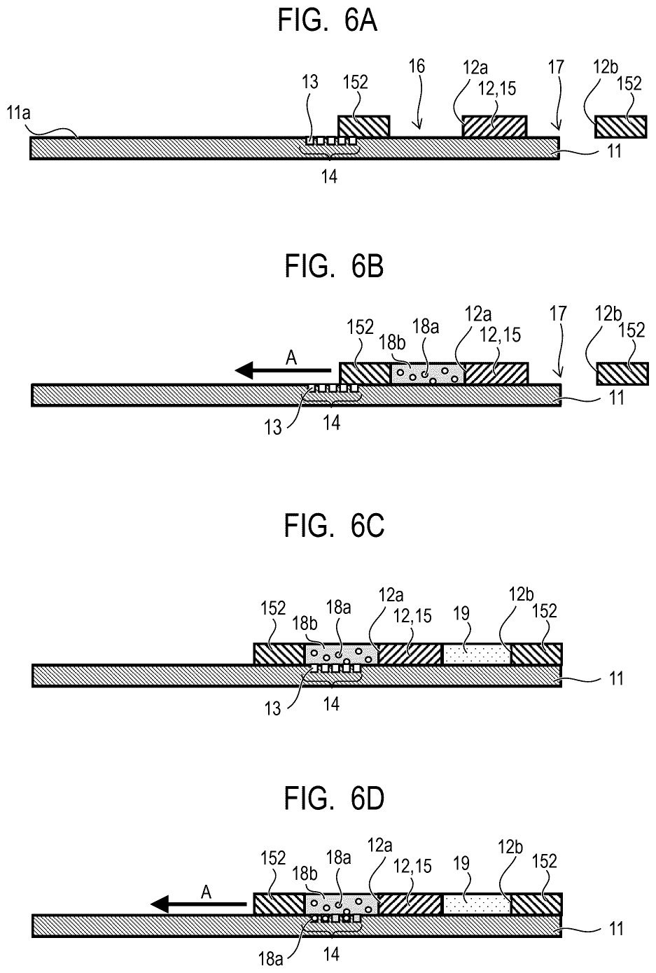

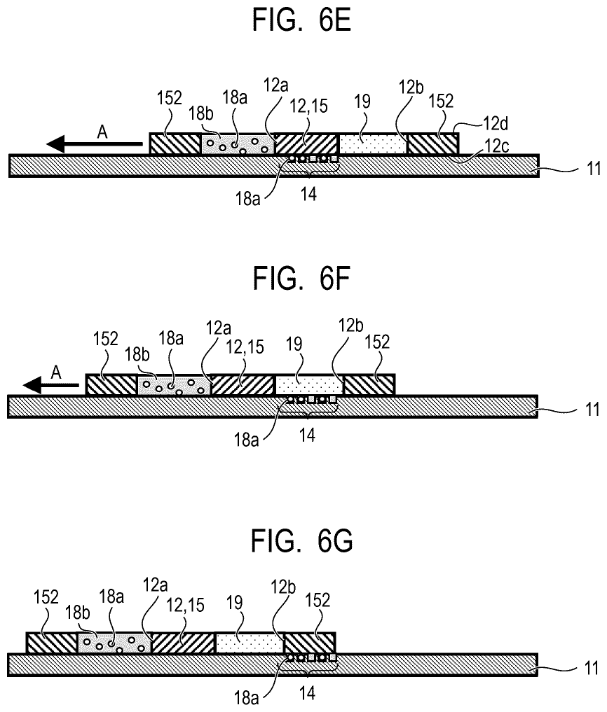

The microwells 13 are wells for storing detection particles 18a dispersed in a dispersion medium 18b. A specific method of storing the detection particles 18a dispersed in the dispersion medium 18b in the microwells 13 is described later with reference to FIG. 3A to FIG. 3G, and FIG. 6A to FIG. 6G.

The wiping member 12 is configured to be capable of moving the dispersion medium 18b present outside the microwells 13 to the outside of the array 14 by being slid in a first direction A under the state of being brought into contact with the upper surface 11a of the base plate 11.

The microwell device according to the present invention preferably further includes a partition wall member 15. Herein, the partition wall member 15 is configured to be capable of being arranged in contact with the upper surface 11a of the base plate 11, and includes at least a first portion 151 extending in the first direction A in the outer side of the array 14 of the upper surface 11a of the base plate 11. Thus, the partition wall member 15 is configured to inhibit the flow of the dispersion medium 18b toward a second direction B perpendicular to the first direction A in the upper surface 11a of the base plate 11.

The partition wall member 15 preferably includes: the two first portions 151 arranged across the array 14; and at least one second portion 152, which connects the corresponding portions of the two first portions 151 to each other and extends in the second direction B. In addition, further, the wiping member 12 is preferably configured to be used under the state of being arranged between the two first portions 151 so as to be capable of sandwiching the array 14 together with the second portion 152. Thus, the dispersion medium 18b containing the detection particles 18a can be introduced into a first region 16 sandwiched between the first surface 12a of the wiping member 12 and the second portion 152, though a specific example of the introduction is described later. Alternatively, a sealing medium 19 can be introduced into a second region 17 sandwiched between the second surface 12b of the wiping member 12 and the second portion 152. Herein, the first surface 12a of the wiping member 12 is a surface configured to be brought into abutment with the dispersion medium 18b to move the dispersion medium 18b in the removing step. In addition, the second surface 12b of the wiping member 12 is a surface configured to be brought into abutment with the sealing medium 19 to move the sealing medium 19 in the sealing step. In addition, when the dispersion medium 18b or the sealing medium 19 is moved with the wiping member 12, the flow of the redundant portion of the dispersion medium 18b or the sealing medium 19 that has passed over the array 14 can be blocked with the second portion 152.

That is, the microwell device includes the partition wall member 15 having the above-mentioned configuration, and hence the staining of an inspection apparatus with the redundant dispersion medium 18b or sealing medium 19 that has flowed out to the outside of the microwell device can be effectively prevented. In addition, the microwell device includes the partition wall member 15, and hence the outflow of the dispersion medium 18b introduced onto the array 14 to a region outside the array 14 can be suppressed, and the efficiency with which the detection particles 18a are packed into the microwells 13 can be improved.

The partition wall member 15 preferably includes the following two second portions 152: the second portion 152 connecting the corresponding end portions of the two first portions 151 on one side to each other; and the second portion 152 connecting the corresponding end portions of the two first portions 151 on the other side to each other. Thus, the flows of both the dispersion medium 18b and the sealing medium 19 in their pushout directions can be blocked.

In the microwell device 10 illustrated in each of FIG. 1 and FIG. 2, the wiping member 12 and the partition wall member 15 are independent of each other, and are configured so that a relative position therebetween may be variable. Meanwhile, the wiping member 12 and the partition wall member 15 may be formed as one body like the microwell devices 20 to 50 illustrated in FIG. 4, FIG. 5, and FIG. 7 to FIG. 9.

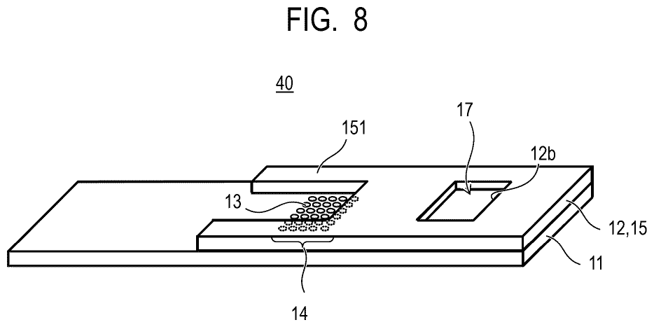

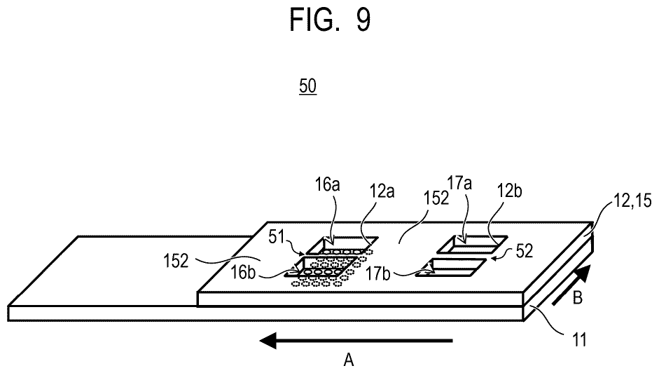

In addition, in each of the microwell devices 10, 20, 40, and 50 illustrated in FIG. 1, FIG. 2, FIG. 4, FIG. 5, FIG. 8, and FIG. 9, the base plate 11 and the partition wall member 15 are configured to be brought into contact with each other on rectangular planes formed by the respective contours of the base plate 11 and the partition wall member 15. However, the entire shape of the microwell device according to the present invention is not limited thereto, and for example, a disc shape like the microwell device 30 illustrated in FIG. 7 is available. In the microwell device 30 illustrated in FIG. 7, the wiping member 12 rotates relative to the base plate 11 about a shaft 31, and hence the dispersion medium 18b present outside the microwells 13 can be moved to the outside of the array 14. That is, in the microwell device 30 illustrated in FIG. 7, the direction in which the wiping member 12 rotates when the dispersion medium 18b present outside the microwells 13 is moved to the outside of the array 14 serves as the first direction A. In the present invention, in the case where the trajectory of the first direction Ain which the wiping member 12 is slid when the dispersion medium 18b present outside the microwells 13 is moved to the outside of the array 14 is not linear, a direction perpendicular to the tangent of the trajectory drawn by the first direction A is defined as the second direction B. That is, when the trajectory drawn by the first direction A is an arc centered on the shaft 31 like the microwell device 30 illustrated in FIG. 7, as illustrated in FIG. 7, the radial direction of a circle centered on the shaft 31 serves as the second direction B.

### <Particle Encapsulation Method>

Subsequently, the respective steps of the particle encapsulation method according to the present invention including using the microwell device described above are described with reference to FIG. 3A to FIG. 3G, and FIG. 6A to FIG. 6G. FIG. 3A to FIG. 3G are schematic sectional views along the first direction A for illustrating the arrangement of the respective elements in the respective steps of the particle encapsulation method including using the microwell device 10 illustrated in each of FIG. 1 and FIG. 2. In addition, FIG. 6A to FIG. 6G are schematic sectional views along the first direction A for illustrating the arrangement of the respective elements in the respective steps of the particle encapsulation method including using the microwell device 20 illustrated in each of FIG. 4 and FIG. 5. In each of FIG. 3A to FIG. 3G, and FIG. 6A to FIG. 6G, to clarify, for example, the wiping member 12, and the dispersion medium 18b and the sealing medium 19 to be moved by the member, the illustration of the first portions 151 of the partition wall member 15 is omitted.

First, an example in which the particle encapsulation method according to the present invention is performed with the microwell device 10 illustrated in each of FIG. 1 and FIG. 2 is described with reference to FIG. 3A to FIG. 3G.

### (Preparing Step)

First, in the preparing step, the microwell device 10 illustrated in each of FIG. 1 and FIG. 2 is prepared.

### (Introducing Step)

Subsequently, in the introducing step, the dispersion medium 18b containing the detection particles 18a is introduced into the array 14.

The introduction of the dispersion medium 18b containing the detection particles 18a into the array 14 may be achieved by directly dropping the dispersion medium 18b containing the detection particles 18a onto the array 14 with a product such as a pipette.

Alternatively, the introduction may be achieved as follows: first, the dispersion medium 18b containing the detection particles 18a is introduced into a region in the upper surface 11a of the base plate 11 where the array 14 is not formed; and subsequently, the dispersion medium 18b containing the detection particles 18a is moved onto the array 14 while the wiping member 12 is slid under the state of being brought into contact with the upper surface 11a of the base plate 11.

Specifically, for example, when the microwell device 10 illustrated in each of FIG. 1 and FIG. 2 is prepared in the preparing step, first, as illustrated in FIG. 3A, the wiping member 12 is arranged at a position sufficiently distant from the array 14. Next, as illustrated in FIG. 3B, the dispersion medium 18b containing the detection particles 18a is introduced into the first region 16 sandwiched between the first surface 12a of the wiping member 12 and the second portion 152. At this time, the position on the upper surface 11a of the base plate 11 into which the dispersion medium 18b containing the detection particles 18a is introduced is set between the array 14 and the first surface 12a. After that, while a state in which the wiping member 12 is brought into contact with the upper surface 11a of the base plate 11 is maintained, the wiping member 12 is slid in the first direction A toward the array 14 to introduce the dispersion medium 18b containing the detection particles 18a onto the array 14 as illustrated in FIG. 3C.

In the introducing step, the dispersion medium 18b containing the detection particles 18a is preferably introduced to such an extent as to cover the entirety of the array 14. When the dispersion medium 18b containing the detection particles 18a is introduced to such an extent as to cover the entirety of the array 14, the dispersion medium 18b containing the detection particles 18a may flow and spread even to a region on the base plate 11 except the array 14. In addition, as a result of the foregoing, the efficiency with which the detection particles 18a are packed into the microwells 13 may reduce, or the staining or contamination of an inspection apparatus may occur. Accordingly, it is preferred that, for example, a partition wall or a groove be arranged on the base plate 11, or instead, to impart the same function, a region corresponding to a partition wall or a groove be made specifically hydrophilic or hydrophobic.

In the particle encapsulation method according to the present invention, the dispersion medium 18b containing the detection particles 18a is introduced not as a flow but as one large mass into the array 14. In addition, an operation for the introduction is mere dropping onto the array 14 or is mere sliding of the wiping member 12 after the introduction of the dispersion medium 18b containing the detection particles 18a into the base plate 11, and hence the detection particles 18a can be introduced into the microwells 13 simply and without unevenness.

### (Packing Step)

Subsequently, in the packing step, the detection particles 18a are packed into the microwells 13. Specifically, as illustrated in FIG. 3D, the detection particles 18a in the dispersion medium 18b are stored in the microwells 13 by, for example, borrowing the power of natural sedimentation or a magnetic force.

The packing of the detection particles 18a in the dispersion medium 18b packed onto the array 14 into the microwells 13 may be achieved by natural sedimentation utilizing the dead weight of the detection particles 18a. However, to further increase the packing ratio of the particles, vibration, a centrifugal force, or the like may be used in combination.

When the detection particles 18a are each a magnetic material, a magnetic field may also be utilized. However, for example, when the magnetic field responsiveness of each of the detection particles 18a each serving as a magnetic material is excessively high, when the intensity of the magnetic field to be applied is excessively large, or when the time period for which the magnetic field is applied is excessively long, the detection particles 18a may be aligned in a height direction along a line of magnetic force by the magnetic field. As a result, concern is raised in that the packing ratio of the detection particles 18a reduces. Accordingly, the intensity and application time of the magnetic field to be applied need to be optimized.

In addition, even when the detection particles 18a are aligned in the height direction along the line of magnetic force by the magnetic field once, a reduction in packing ratio is preferably prevented by loosening the detection particles 18a through the weakening of the magnetic field to be applied or the setting of the time period for which no magnetic field is applied.

In addition, to more efficiently pack the detection particles 18a into the microwells 13, a deaeration operation is preferably used in combination. An environment in which the detection particles 18a are efficiently packed into the microwells 13 can be established by forcibly performing a gas-liquid exchange between air in the microwells 13 and the dispersion medium 18b containing the detection particles 18a through the deaeration operation. For example, a method including placing and leaving the base plate 11 including the array 14 or the entirety of the microwell device 10 under a reduced-pressure environment may be suitably used as a method for the deaeration. Specifically, for example, a method including leaving the base plate 11 including the array 14 or the entirety of the microwell device 10 in a vacuum desiccator at about 0.1 atm for about 30 seconds is available.

When the detection particles 18a encapsulated in the microwells 13 by the particle encapsulation method according to the present invention are subjected to inspection, from the viewpoint of improving inspection accuracy, in the packing step, the one detection particle 18a is preferably packed on average into each of the microwells 13.

### (Removing Step)

Next, in the removing step, the dispersion medium 18b present outside the microwells 13 is removed. Specifically, in order that the first surface 12a serving as the surface on which the wiping member 12 is brought into abutment with the dispersion medium 18b may pass over the array 14, the wiping member 12 is slid in the first direction A again while being brought into abutment with the base plate 11. Thus, as illustrated in FIG. 3E, the dispersion medium 18b present outside the microwells 13 is moved to a position sufficiently distant from the array 14.

When not all the detection particles 18a in the dispersion medium 18b introduced into the array 14 are packed into the microwells 13, the detection particles 18a and the dispersion medium 18b serving as a dispersion medium for the detection particles 18a are present on the array 14. In addition, when all the detection particles 18a in the dispersion medium 18b introduced into the array 14 are packed into the microwells 13, the dispersion medium 18b serving as a dispersion medium for the detection particles 18a is present on the array 14. The dispersion medium 18b present outside the microwells 13 refers to both of the foregoing.

The dispersion medium 18b present outside the microwells 13 is removed as follows: while the wiping member 12 is slid on the base plate 11, the medium is moved to the region of the base plate 11 where the array 14 is not formed. At this time, when the detection particles 18a in the dispersion medium 18b are sedimented on the base plate 11 where the microwells 13 in the array 14 are absent, the detection particles 18a are expected to be moved to enter the microwells 13 in the removing step, and hence their packing ratio can be increased.

In addition, when the deaeration operation is used in combination, fine air bubbles that have surfaced from the insides of the microwells 13 in the removing step are removed together. In the related art, the following approach may be used: under a state in which the fine air bubbles are present as a result of the deaeration operation, the dispersion medium 18b containing the detection particles 18a is pushed away with a liquid sealing medium through use of a flow cell. In this case, a flow that avoids the air bubbles is formed to preclude uniform outflow of the dispersion medium 18b, and as a result, the microwells 13 are not sealed uniformly and without unevenness in some cases. In addition, when the liquid sealing medium is flowed vigorously or flowed in a large amount to push out the air bubbles, concern is raised about the contamination of an inspection apparatus with the dispersion medium 18b or the sealing medium 19 that has appeared to the outside of the microwell device 10.

However, in the particle encapsulation method according to the present invention, the sliding of the wiping member 12 removes the dispersion medium 18b not as a flow but as one large mass from above the array 14. In addition, the foregoing operation is to merely slide the wiping member 12 after the introduction of the dispersion medium 18b into the array 14 to pack the detection particles 18a into the microwells 13, and hence the dispersion medium 18b can be removed simply and without unevenness. In addition, the mixing of air bubbles at the time of the sealing of the microwells can be suppressed.

When the detection particles 18a are packed by using a magnetic field, as described above, the detection particles 18a may be aligned in a height direction along a line of magnetic force depending on the magnetic field responsiveness of each of the detection particles 18a, or the intensity or time length of the magnetic field to be applied. When the sliding operation is performed under the state, concern is raised in that the detection particles 18a are liable to enter a space between a cover plate and the base plate to hinder the step of removing the liquid medium and the sealing step to be described later. Accordingly, it is preferred that the magnetic field to be applied be weakened or no magnetic field be applied before the sliding operation.

### (Sealing Step)

Subsequently, in the sealing step, the microwells 13 are sealed with the sealing medium 19. Specifically, first, as illustrated in FIG. 3E, the sealing medium 19 is introduced into the second region 17 sandwiched between the second surface 12b of the wiping member 12 and the second portion 152 of the partition wall member 15. At this time, the position on the upper surface 11a of the base plate 11 into which the sealing medium 19 is introduced is set between the array 14 and the second surface 12b. Subsequently, the wiping member 12 is slid in a direction opposite to the first direction A, in other words, toward the array 14 under the state of being brought into contact with the upper surface 11a of the base plate 11, to thereby move the sealing medium 19 onto the microwells 13 to seal the microwells as illustrated in FIG. 3F. In other words, in the example illustrated by the microwell device 10, the first surface 12a on which the wiping member 12 is brought into abutment with the dispersion medium 18b, and the second surface 12b on which the wiping member 12 is brought into contact with the sealing medium 19 are surfaces different from each other.

In the sealing step, for example, when the sealing medium 19 is a liquid, first, the sealing medium 19 may be directly dropped onto the array 14 with a product such as a pipette. In addition, as described above, the sealing medium 19 may be moved onto the array 14 with the wiping member 12 after having been introduced into a region on the base plate 11 where the array 14 is not formed. Thus, the layer of the sealing medium 19 is formed on the microwells 13 to seal the microwells 13. After that, as illustrated in FIG. 3G, the wiping member 12 is further gently slid to move the sealing medium 19 introduced onto the array 14 to the region on the base plate 11 where the array 14 is not formed. At this time, attention is paid so that the wiping member 12 may not be excessively pressed against the base plate 11. Thus, a thin film of the sealing medium 19 is formed on the microwells 13, and hence the microwells 13 can be sealed.

When the sealing medium 19 is a solid such as a cover glass, the microwells 13 can also be sealed by superimposing the sealing medium 19 on the array 14. At this time, the sealing medium 19 may be superimposed from above the array 14 in a descending manner, or may be moved and superimposed onto the array 14 by being slid on the base plate.

In addition, in the sealing step, two operations may be combined as follows: the microwells are sealed with the liquid sealing medium 19 once; and then the microwells are sealed with the solid sealing medium 19 again.

Next, an example in which the particle encapsulation method according to the present invention is performed with the microwell device 20 illustrated in each of FIG. 4 and FIG. 5 is described with reference to FIG. 6A to FIG. 6G.

### (Preparing Step)

First, in the preparing step, the microwell device 20 illustrated in each of FIG. 4 and FIG. 5 is prepared.

### (Introducing Step)

Subsequently, first, as illustrated in FIG. 6A, the wiping member 12 is arranged on the upper surface 11a of the base plate 11 so that the first region 16 sandwiched between the second portion and the first surface 12a of the wiping member 12 may not overlap the array 14. Next, as illustrated in FIG. 6B, the dispersion medium 18b containing the detection particles 18a is introduced into the first region 16. Subsequently, as illustrated in FIG. 6C, the dispersion medium 18b is introduced into the array 14 as follows: the wiping member 12 is slid in the first direction A while a state in which the member is brought into contact with the upper surface 11a of the base plate 11 is maintained, to thereby cause the first region 16 to cover the array 14.

### (Packing Step)

Next, as illustrated in FIG. 6D, the detection particles 18a in the dispersion medium 18b are stored in the microwells 13 by, for example, borrowing the power of natural sedimentation or a magnetic force.

### (Removing Step)

As illustrated in FIG. 6C or FIG. 6D, the sealing medium 19 is introduced into the second region 17 under a state in which the wiping member 12 is arranged so that the first region 16 may cover the array 14. Then, the wiping member 12 is slid in the first direction A again while being brought into contact with the base plate 11, to thereby move the dispersion medium 18b introduced into the first region 16 to a region on the base plate 11 where the array 14 is not formed as illustrated in FIG. 6E. Thus, the dispersion medium 18b present outside the microwells 13 is removed. At this time, the sealing medium 19 introduced into the second region 17 is simultaneously removed (FIG. 6E).

At this stage, when the wiping member 12 also functions as the sealing medium 19 to seal the microwells 13, the removing step and the sealing step are simultaneously performed, and hence the sealed detection particles 18a can be subjected to inspection or measurement.

### (Sealing Step)

From the state illustrated in FIG. 6E, the wiping member 12 is continuously slid as it is in the first direction A while a state in which the member is brought into contact with the base plate 11 is maintained. Then, as illustrated in FIG. 6F, the wiping member 12 is arranged so that the sealing medium 19 introduced into the second region 17 may cover the array 14. Thus, the microwells 13 can be sealed with the sealing medium 19 (FIG. 6F).

Further, the wiping member 12 is continuously slid as it is in the first direction A while a state in which the member is brought into contact with the base plate 11 is maintained. Thus, as illustrated in FIG. 6G, the redundant sealing medium 19 on the array 14 is removed by being moved with the wiping member 12 to a region on the base plate 11 where the array 14 is not arranged. After that, as illustrated in FIG. 6G, a state in which the microwells 13 are sealed with both of a thin layer of the sealing medium 19 and the bottom surface of the wiping member 12 may be established. Alternatively, the microwells 13 may be sealed only with the sealing medium 19 by further sliding the wiping member 12 in the first direction A to move the member to a position distant from the top of the array 14.

When the wiping member 12 is slid as described above, even in, for example, the case where the array 14 has a large area of more than 1 cm², the detection particles 18a can be encapsulated in the microwells 13 simply, uniformly, and with high efficiency.

In the process described with reference to FIG. 6A to FIG. 6G, all of the four steps, that is, the introducing step, the packing step, the sealing step, and the removing step are performed by sliding the wiping member 12. However, the present invention is not limited thereto. When one or more steps including the removing step out of the above-mentioned four steps can be performed by sliding the wiping member 12, the particles can be encapsulated more simply than before.

The introducing step may be performed when the wiping member 12 is present at a position illustrated not in FIG. 6B but in FIG. 6C with respect to the base plate 11. In this case, the introducing step does not include any operation of sliding the wiping member 12.

In addition, an example in which the sealing medium 19 is introduced into the second region 17 when the wiping member 12 is present at the position illustrated in FIG. 6C with respect to the base plate 11 has been described. However, the present invention is not limited thereto. For example, when the base plate 11 is present below the second region 17 in the arrangement illustrated in FIG. 6B, the sealing medium 19 may be introduced into the second region 17 at the time of the arrangement. In addition, the sealing medium 19 may be introduced into the second region 17 simultaneously with the performance of the introducing step when the wiping member 12 is present at the position illustrated in FIG. 6C with respect to the base plate 11 as described above. In addition, the sealing medium 19 may be introduced into the second region 17 when the wiping member 12 and the base plate 11 are arranged as illustrated in FIG. 6E or FIG. 6F.

Next, the respective elements according to the present invention are further described.

### (Base Plate)

A material for forming the base plate is preferably selected from, for example, a cycloolefin polymer, a cycloolefin copolymer, silicon, glass, an ABS resin, a polycarbonate resin, an acrylic resin, polyvinyl chloride, a polystyrene resin, a polyethylene resin, a polypropylene resin, polyvinyl acetate, polyethylene terephthalate (PET), and polyethylene naphthalate (PEN). In consideration of the fact that the detection particles encapsulated in the microwells arranged in the base plate are subjected to inspection or measurement after the encapsulation, the base plate is preferably formed of a material having translucency. In addition, the material for forming the base plate is preferably free of autofluorescence. Further, in consideration of processability for forming a microwell array, the cycloolefin polymer, the cycloolefin copolymer, or the acrylic resin is more preferably selected.

The base plate includes a region where the array formed of the microwells is formed and a flat region where nothing is formed. In addition, a hydrophilic liquid is often suitably used as a dispersion medium for the detection particles. Accordingly, to increase the ratio at which the detection particles are packed into the microwells formed in the base plate, at least the region where the array is formed is preferably subjected to hydrophilic treatment.

As described above, when the microwell device includes the partition wall member, the ratio at which the detection particles are packed into the microwells can be increased, and the staining of an inspection apparatus can be prevented. However, when the microwell device does not include the partition wall member, a site on the base plate where the partition wall member may be arranged is preferably subjected to such treatment as described below: such chemical treatment that the site becomes hydrophilic or hydrophobic; or physical treatment such as the arrangement of a groove.

### (Wiping Member and Partition Wall Member)

As described above, the wiping member is configured to be capable of moving the dispersion medium present outside the microwells to the outside of the array by being slid in the first direction under the state of being brought into contact with the upper surface of the base plate.

The width of the first surface, on which the wiping member is brought into abutment with the dispersion medium for the detection particles, in the second direction is preferably larger than the width of the array in the second direction. The term “second direction” as used herein refers to a direction perpendicular to the first direction serving as the direction in which the wiping member is slid in the removing step. Thus, the removing step can be performed by one sliding operation.

The wiping member functions as a wiper by being slid on the base plate, and at least the removing step out of the four steps, that is, the introducing step, the packing step, the sealing step, and the removing step is performed by the sliding operation.

In the operation of sliding the wiping member, the wiping member is slid while a force is applied in such a direction that the member is pressed against the base plate. Accordingly, to smoothly slide the wiping member so that the member may not be caught by the base plate, the edge of the third surface of the wiping member to be brought into contact with the base plate is preferably subjected to moderate R-processing.

In addition, to slide the wiping member while bringing the member and the base plate into close contact with each other like the water pasting of a film, a medium (a water-soluble medium or a hydrophobic medium) for improving slidability may be arranged between the wiping member and the base plate. For example, the same medium as the dispersion medium for the detection particles or the sealing medium may be used as the medium for improving slidability.

For example, to inspect a plurality of samples or a plurality of items with one array, the wiping member may include a partition like, for example, the microwell device 50 illustrated in FIG. 9. Herein, the partition has a function of dividing the microwells for forming the array into two or more groups. That is, the wiping member 12 may include one or more first partitions 51 configured to extend from the first surface 12a in the first direction A to inhibit the movement of the dispersion medium 18b toward the second direction B perpendicular to the first direction A. In addition, the wiping member 12 may include one or more second partitions 52 configured to extend from the second surface 12b in the first direction A to inhibit the movement of the sealing medium 19 toward the second direction B. The second partitions 52 are preferably arranged at corresponding positions as those of the first partitions 51 in the second direction B with respect to the wiping member 12. That is, the first partitions 51 and the second partitions 52 preferably pass through the same trajectory when the wiping member 12 slides on the base plate 11.

In the microwell device 50 illustrated in FIG. 9, the first region 16 sandwiched between the first surface 12a and the second portion 152 is divided into two third regions 16a and 16b defined by the first partition 51 and the first surface 12a. Accordingly, when the microwell device 50 is used, the introducing step may include introducing the dispersion medium 18b containing the detection particles 18a into the third regions 16a and 16b.

In addition, similarly, in the microwell device 50 illustrated in FIG. 9, the second region 17 sandwiched between the second surface 12b and the second portion 152 is divided into two fourth regions 17a and 17b defined by the second partition 52 and the second surface 12b. Accordingly, when the microwell device 50 is used, the sealing step may include introducing the sealing medium 19 into the fourth regions 17a and 17b.

In the case where the wiping member includes the first partition and the second partition as described above, when the array has a sufficiently wide size, a plurality of samples or a plurality of items can be inspected. In addition, the microwell device may be used while sealing media different from each other are introduced into the respective fourth regions divided from each other by the second partition.

As described in the description of the particle encapsulation method including using the microwell device 20 with reference to FIG. 6A to FIG. 6G, the wiping member 12 may have a function as the sealing medium 19. That is, the third surface 12c of the wiping member 12 to be brought into contact with the base plate 11 may include a sealing surface having a size enough to cover the array 14. At this time, the sealing step may include, for example, arranging the wiping member 12 at, for example, a position illustrated in FIG. 6E with respect to the base plate 11 to cover the array 14 with the sealing surface.

Further, for example, when the base plate 11 is formed of a material having translucency, and the sealing surface is coated with a metal, in the case where the microwells 13 are sealed with the coated surface of the third surface of the wiping member 12, inspection including finally utilizing light can be suitably performed. Specifically, for example, emitted light in the microwells 13 is reflected by the surface of the metal coating the sealing surface, and as a result, the efficiency with which the emitted light is extracted is improved. Thus, detection sensitivity can be improved.

In addition, when the coating of the sealing surface with the metal is performed by vapor deposition, an adhesive force between the metal and the substrate (sealing surface) is weak, and hence concern is raised about the peeling of a thin film of the metal that has been deposited from the vapor in the course of the sliding of the wiping member 12. Accordingly, not the sealing surface arranged on the third surface 12c on which the wiping member 12 is brought into direct contact with the base plate 11 but a fourth surface opposite to the third surface 12c of the wiping member 12 may be coated with the metal. That is, a fourth surface 12d opposite to the third surface 12c of the wiping member 12 to be brought into contact with the base plate 11 may include a metal-coated surface that faces the sealing surface and has a size enough to cover the sealing surface. In this case, the microwells 13 are sealed by covering the array 14 with the sealing surface in the sealing step. In addition, although light cannot be directly reflected in the range of the thickness of the wiping member 12, the efficiency with which emitted light is extracted can be improved as compared to that in the case where the wiping member 12 is free of any metal-coated surface.

The wiping member is preferably configured to be used together with the partition wall member including the two first portions, and to be used under the state of being connected to each of the two first portions. Thus, a region formed by the first surface of the wiping member and the first portions of the partition wall member, and a region formed by the second surface of the wiping member and the first portions of the partition wall member can be completely divided from each other across the wiping member itself. Thus, no concern is raised in that the dispersion medium and the sealing medium mix with each other, and hence it becomes easier to operate the microwell device.

In the microwell device according to the present invention, when no structure having a function as the partition wall member is arranged on the upper surface 11a of the base plate 11, the microwell device preferably includes the partition wall member.

Although the partition wall member includes at least the first portion extending in the first direction, the portions being configured to inhibit the flow of the dispersion medium toward the second direction, the length of the first portion in the first direction is preferably longer than the length of the array in the first direction.

In each of FIG. 1, FIG. 2, FIG. 4, FIG. 5, and FIG. 7 to FIG. 9, the following example is illustrated: the shape of the partition wall member 15 is defined by the first portion 151 linearly extending in the first direction A in which the wiping member 12 is slid, and the second portion 152 linearly extending in the second direction B perpendicular to the first direction. However, the shape of the partition wall member 15 is not limited thereto. That is, the partition wall member 15 only needs to have a structure, which functions as a wall for preventing the staining of an inspection apparatus due to the flow of the dispersion medium 18b or the sealing medium 19 to the outside of the array 14, and the shape of the structure functioning as a wall is not particularly limited. For example, the shapes of the first region 16 and the second region 17 are also not particularly limited, and the contours thereof may each be a quadrangle or a circle. It is particularly preferred that when the first region 16 and the second region 17 overlap the array, the first region 16 and the second region 17 each have an area enough to completely cover the array.

Although the partition wall member may include a plurality of members, the member preferably includes one member in consideration of cost and the complexity of its structure. Further, it is more preferred that the partition wall member and the wiping member be integrated with each other to form one member.

The microwell device 20 illustrated in each of FIG. 4 and FIG. 5 is an example of a microwell device whose partition wall member and wiping member form one member. The microwell device 20 has such a form that holes (the first region 16 and the second region 17) penetrating the one member formed by integrating the partition wall member and the wiping member with each other are opened. In the microwell device 20, it is walls in the direction in which the wiping member is slid out of the inner walls of the holes that function as the first surface 12a and the second surface 12b. In other words, in the example illustrated in FIG. 4, quadrangular holes are arranged, and walls inside the holes function as the surfaces. In FIG. 4, as an example, the following two holes are arranged in the member: a hole serving as the first region 16 into which the dispersion medium 18b containing the detection particles 18a is to be introduced; and a hole serving as the second region 17 into which the sealing medium 19 is to be introduced. However, the present invention is not limited thereto. For example, to reduce the difficulty of processing, the number of the holes to be arranged in the member may be one. The microwell device 40 illustrated in FIG. 8 is an example of a case in which the number of the holes arranged in the one member formed by integrating the partition wall member and the wiping member with each other is one. The wiping member 12 (partition wall member 15) of the microwell device 40 has such a shape that in the wiping member 12 (partition wall member 15) illustrated in each of FIG. 4 and FIG. 5, the second portion for forming a wall of one hole on a side in the first direction A is removed.

Such structures functioning as walls as exemplified by the first portion and second portion of the partition wall member each preferably have a height enough to prevent the overflow of the dispersion medium containing the detection particles or the liquid sealing medium at the time of the introduction of the dispersion medium or the sealing medium. When the structures of the partition wall member functioning as walls each have a sufficient height, in the case where the first region or the second region is a closed space, the storage of the dispersion medium or the sealing medium in such region eliminates concern about the wet spread thereof due to the lapse of time or vibration. In addition, when the microwell device is tilted, the dispersion medium or the sealing medium in the first region or the second region can be suppressed from spilling.

A material, which is free of a component that is eluted in a solvent in the dispersion medium containing the detection particles or the sealing medium, is preferably selected as a material for forming each of the wiping member and the partition wall member in consideration of the fact that the members are brought into contact with the dispersion medium and the sealing medium. The material for forming each of the wiping member and the partition wall member may be specifically selected from, for example, the materials listed in the description of the base plate.

### (Microwells and Array)

A known production method, such as photolithography, microinjection molding, or nanoimprinting, may be selected for the formation of the array formed of the microwells. The shape of the contour of the entirety of the array is not particularly limited, and may be a quadrangle or a circle. When a plurality of samples or a plurality of items are inspected as described above, a quadrangle is preferred because the respective steps in the particle encapsulation method can be efficiently performed by the operation of sliding the wiping member.

Although the shape of each of the microwells for forming the array is also not particularly limited, the shape is preferably, for example, a column, a cone, or a truncated cone in terms of ease of processing. The shape of each of the microwells is more preferably a truncated cone in consideration of, for example, the fact that when a material for forming the microwells is removed from a mold, the defects of the microwells due to the contact of the mold with a substrate hardly occur, and the ease with which the detection particles enter the microwells.

The minimum aperture of the opening portion of each of the microwells and the depth of the microwell are equal to or more than the diameter of each of the detection particles. Although one or more detection particles may enter each of the microwells, when the number of the detection particles entering each microwell is large, the total number of the particles to be required increases to increase inspection cost, and contrivance to improve detection accuracy is required in some cases. Accordingly, the number of the detection particles entering each of the microwells is preferably 1 or more and 4 or less, more preferably 1. In view of the foregoing, the maximum length of the opening diameter of each of the microwells, and the depth of the microwell are each preferably 1.0 times or more and less than 2.0 times as large as the average particle diameter of the detection particles. In addition, the maximum length of the opening diameter of each of the microwells is more preferably 1.5 times or more and less than 2.0 times as large as the average particle diameter of the detection particles. The minimum aperture of each of the microwells is specifically, for example, 1.5 μm or more and less than 10 μm. In addition, the depth of each of the microwells is specifically, for example, 1.0 μm or more and less than 10 μm.

When a distance between the microwells in the array is excessively short, it becomes difficult to separate signals at the time of the observation of the detection particles or to perform the processing itself of the microwells. However, when the distance is excessively long, the number of signals that can be acquired reduces in the case where the area of the microwell array remains constant. Accordingly, the distance between the centers of the microwells is preferably 5 μm or more and 15 μm or less.

### (Detection Particles)

The average particle diameter of the detection particles is preferably 1 μm or more and 5 μm or less. Thus, efficient packing of the particles into the microwells, and an increase in density of the array can be achieved. The term “average particle diameter” as used herein refers to a value measured by using observation with an electron microscope or a dynamic light scattering method.

The detection particles may each include any one of organic matter and inorganic matter, and various materials may be combined. In consideration of the step of packing the detection particles into the microwells, and the bonding of a molecule for capturing a target substance to be described later, the detection particles are preferably formed as described below. That is, the detection particles are each preferably configured to include an inorganic layer having a heavy specific gravity on a core side, and to include, as an outermost shell, an organic layer to which the molecule for capturing the target substance is easily bonded.

A material for forming the inorganic layer is, for example, silica, alumina, iron oxide, iron, zirconia, or titanium oxide. The core of each of the detection particles may be formed from a single material, or may be formed in such a form that the layer of organic matter is covered with the inorganic layer.

As commercially available particles that may be used in the present invention, there are given, for example, Magnosphere (trademark) MS300, Magnosphere (trademark) MS160, PureProteome (trademark) Nickel Magnetic Beads, magnetic particles to be used in Simoa (trademark) Homebrew Assay Kit (Quanterix), Polybead (trademark) Microspheres, and HIPRESICA (trademark) manufactured by Ube Exsymo Co., Ltd.

Detection particles each containing such a material as described below as a constituent material are preferably used as the detection particles: a paramagnetic material, such as iron, nickel, or magnetite; a ferromagnetic material; or a superparamagnetic material. However, detection particles each containing a material except the foregoing may be used. When magnetic particles are used, the application of a magnetic field facilitates not only the packing of the detection particles into the microwells described above but also the purification of the detection particles that have each captured the target substance after a reaction step of causing each of the detection particles to capture the target substance to be described later.

In addition to the detection particles that have each captured the target substance, the detection particles that have each not captured the target substance may be mixed in the detection particles to be packed and encapsulated in the microwells. For example, detection particles each having bonded thereto a molecule for specifically capturing the target substance may be used as the detection particles that each specifically capture the target substance. The molecule for specifically capturing the target substance may be bonded to a modifying group present on the surface of each of the detection particles through, for example, a linker. The molecule for specifically capturing the target substance may be bonded to, for example, an amino group present on the surface of each of amino group-modified detection particles by covalent bonding through a crosslinking agent having N-hydroxysuccinimide or the like. The molecule for specifically capturing the target substance only needs to be selected in accordance with the target substance, and for example, a protein, an antibody, or a nucleic acid may be used. It is preferred that 100,000 or more molecules for specifically capturing the target substance be bonded to one detection particle. In, for example, the case where the target-capturing molecule is an antibody, the dissociation constant of the antibody is of about a nanomolar order, but when the above-mentioned configuration is adopted, the concentration of the target-capturing molecules when the detection particles and the target substance are caused to react with each other can be made sufficiently high.

The number of the detection particles to be introduced into the array is preferably equal to or more than the total number of the microwells for forming the array, and an index of the upper limit of the number of the detection particles to be introduced may be determined by a relationship between the sizes of the microwells and the detection particles. For example, when only one detection particle can be stored in one microwell, the upper limit of the number of the detection particles to be introduced may be made equal to the total number of the microwells for forming the array. In addition, for example, when such a size relationship that three detection particles can be stored in one microwell is established, the upper limit of the number of the particles to be introduced only needs to be made three times as large as the total number of the microwells for forming the array.

### (Target Substance)

The term “target substance” refers to a molecule serving as a detection object, that is, herein, a molecule whose detection is attempted as follows: each of the detection particles is caused to capture the molecule. Examples of the target substance include: biomolecules, such as a protein, an enzyme, a nucleic acid, a peptide, a lipid metabolite, and a sugar; and viral particles themselves.

The enzyme includes, for example, all kinds of enzymes that extracellular vesicles have on their vesicle surfaces or in themselves. Examples of such enzyme may include: hydrolases typified by various proteases including a MMP family, such as matrix metalloproteinase (MMP), a disintegrin and metalloproteinase (ADAM), and ADAM with thrombospondin motifs (ADAMTS), matriptase, β-secretase, endothelin-converting enzyme (ECE), calpain, dipeptidyl peptidase-4 (DPPIV), angiotensin-converting enzyme 1 (ACE1), and angiotensin-converting enzyme 2 (ACE2), various esterases including acetylcholinesterase (AChE), autotaxin, lipase, phospholipase, and phosphatase, and various glycosidases including β-galactosidase; oxidoreductases typified by various oxidases including monoamine oxidase (MAO), various peroxidases including myeloperoxidase (MPO), catalase, and superoxide dismutase; transferases typified by various acetylases/deacetylases including histone acetyltransferase (HAT) and histone deacetylase (HDAC), kinase, protein kinase, and saccharyltransferase; and isomerases typified by various cis-trans isomerases including Pin1 (peptidylprolylisomerase: PPIase), racemase, and mutase.

### (Dispersion Medium)

As the dispersion medium, which contains the detection particles and is used for packing the detection particles into the microwells, a medium appropriately selected from various media in accordance with the detection particles to be used may be used. Of those, a hydrophilic liquid medium may be suitably used as the dispersion medium in many cases. The hydrophilic liquid medium that may be used as the dispersion medium is, for example, at least one selected from the group consisting of: water; a hydrophilic alcohol; a hydrophilic ether; a ketone; a nitrile-based solvent; dimethyl sulfoxide; and N,N-dimethylformamide, or a mixture containing two or more of these media. Examples of the hydrophilic alcohol include ethanol, methanol, propanol, and glycerin. Examples of the hydrophilic ether include tetrahydrofuran, polyethylene oxide, and 1,4-dioxane. Examples of the ketone include acetone and methyl ethyl ketone. An example of the nitrile-based solvent is acetonitrile.

The dispersion medium is used while containing the detection particles that have each captured the target substance and the detection particles that have each not captured the target substance. In addition to the foregoing, the dispersion medium may further contain, for example, a substance for specifically detecting the target substance captured by each of the detection particles, a standard fluorescent substance for recognizing a liquid droplet or a microwell, a blocking material, a buffer, or a surfactant.

The substance for specifically detecting the target substance captured by each of the detection particles is preferably, for example, a fluorescent substrate that is degraded by a predetermined enzyme, which is bonded to the target substance captured by each of the detection particles or to a molecule specifically bonded thereto, to liberate a fluorescent substance. The molecule specifically bonded to the target substance may be, for example, a secondary antibody or a nucleic acid.

A reporter molecule whose emission intensity may be enlarged by a change in structure thereof caused by enzyme activity may be, for example, a molecule labeled with a fluorescent substance and a quenching substance. Such reporter molecule may be, for example, the following molecule (FRET substrate): the reporter molecule has the fluorescent substance and the quenching substance on both of its terminals or modifiable amino acid residues in itself, and contains, between the substances, a peptide serving as a substrate for a target enzyme. In the FRET substrate, before enzyme cleavage, the fluorescent substance and the quenching substance are sufficiently close to each other, and hence the fluorescent substance is quenched. However, when the peptide is cleaved by the enzyme activity, the quenching by the quenching substance is released, and hence the fluorescent substance emits fluorescence. The use of the FRET substrate as the reporter molecule enables the observation of the presence or absence of the target enzyme as a large change in brightness of the fluorescence. The number of the residues of the peptide in the FRET substrate is preferably 3 or more in terms of reactivity, and is preferably 30 or less in terms of quenching effect. In addition, a reporter molecule that may be transformed from a nonfluorescent substance to a fluorescent substance by a change in chemical structure thereof caused by the enzyme activity is preferred because a large brightness change is obtained. A reporter molecule of a commercially available kit may be appropriately used as such reporter molecule.

In addition, the reporter molecule may be synthesized in a custom manner as follows: based on the substrate sequence of the enzyme serving as a target, the fluorescent substance and the quenching substance are combined on both the terminals of the substrate sequence or in the substrate sequence.

A dye having a suitable emission wavelength may be selected as the standard fluorescent substance for recognizing a liquid droplet or a microwell from, for example, an Alexa Fluor (trademark) dye and a DyLight (trademark) dye. When the former dye and the latter dye are used in combination, dyes having different emission wavelengths are preferably selected so that the liquid droplet or the microwell may be easily observed.

The blocking material has the following function: when each of the detection particles and a capturing protein are bonded to each other, the material fills a portion to which the capturing protein has not been bonded out of the linker-bonded portion of the detection particle. For example, when an amino group of the capturing protein and each of the carboxyl groups of the detection particles are subjected to a condensation reaction through use of NHS/WSC, the detection particles may have unreacted carboxyl groups after the reaction. In view of the foregoing, the carboxyl group that has not been bonded to the capturing protein may be caused to react with, for example, ethanolamine serving as a blocking agent or PEG having an amino group. A known material may be used as the blocking material, and examples thereof include Blockmaster (trademark) CE510, CE210, DB1130, and PA1080.

Examples of the buffer include a Tris-based buffer and a HEPES-based buffer.

The surfactant may be used in the stabilization of the dispersion of the detection particles or the stabilization of a protein. Examples of the surfactant include Brij 35, Brij 58, Tween 20, Tween 80, NP 40, Triton X-100, and Triton X-114. Specific examples of the surfactant include Binding buffer (20 mM Tris-HCl (pH: 7.6), 100 mM KCl, 5 mM MgCl₂, 1 mM DTT, 5% glycerol, 50 μg/mL heparin), NE Buffer (trademark) 2.1 (10 mM Tris-HCl, 50 mM NaCl, 10 mM MgCl₂, 100 μg/mL bovine serum albumin (BSA), pH: 7.9), and FZ Buffer (20 mM HEPES, 60 mM NaCl, 6 mM MgCl₂, pH: 6.8).

### (Sealing Medium)

When the sealing medium is a liquid, the use of a hydrophobic liquid medium as the sealing medium can suppress the sealing medium and the dispersion medium from mixing with each other in the case where a hydrophilic liquid medium is used as the dispersion medium. For example, at least one selected from the group consisting of: a saturated hydrocarbon; an unsaturated hydrocarbon; an aromatic hydrocarbon; a silicone oil; a fluorine-based oil; perfluorocarbon; a halogen-based solvent; and a hydrophobic ionic liquid, or a mixture containing the above-mentioned medium may be preferably used as the hydrophobic liquid medium.

Examples of the saturated hydrocarbon include an alkane and a cycloalkane. Examples of the alkane include decane and hexadecane. An example of the unsaturated hydrocarbon is squalene. Examples of the aromatic hydrocarbon include benzene and toluene. An example of the fluorine-based oil is SR-X Sealing Oil. An example of the perfluorocarbon is Fluorinert (trademark) FC40 (manufactured by Sigma-Aldrich Co. LLC). Examples of the halogen-based solvent include chloroform, methylene chloride, and chlorobenzene. The term “hydrophobic ionic liquid” refers to an ionic liquid that does not dissociate at least in water, and the liquid is, for example, 1-butyl-3-methylimidazolium hexafluorophosphate. The term “ionic liquid” refers to a salt that is present in a liquid form at room temperature.

When the sealing medium is a solid, as described above, for example, the wiping member may be used as the sealing medium. A glass, a metal, or the like may be selected as a material for forming the solid sealing medium, and these materials may be combined.

### <Target Substance Detection Method>

A target substance detection method according to the present invention includes a reaction step, a particle-encapsulating step, and a detecting step. The reaction step is a step of causing detection particles that each specifically capture a target substance and the above-mentioned target substance to react with each other. The particle-encapsulating step is a step of performing the particle encapsulation method according to the present invention, which has heretofore been described, with the above-mentioned detection particles after the above-mentioned reaction step. In addition, the detecting step is a step of detecting the detection particles that have each captured the above-mentioned target substance out of the above-mentioned detection particles stored in the respective microwells described above.

### (Reaction Step)

The reaction step is a step of causing the target substance to react with the detection particles, and may be achieved by, for example, mixing a solution containing the detection particles and a solution containing the target substance to cause the solutions to react with each other. For example, a known molecular recognition reaction or intermolecular interaction, such as an antigen-antibody reaction, a streptavidin-biotin reaction, or complementary bonding of a nucleic acid, may be suitably used as the reaction to be performed in the reaction step.

### (Particle-Encapsulating Step)

In the particle-encapsulating step, the respective steps in the above-mentioned particle encapsulation method may be similarly performed.

### (Detecting Step)

The detecting step is a step of detecting whether or not the detection particle that has captured the target substance is packed into each of the microwells after the particle-encapsulating step.

A method of detecting whether or not the detection particle that has captured the target substance is stored in each of the microwells is, for example, a method including utilizing fluorescence detection. The method may specifically include, for example, incubating the base plate having the microwells to detect fluorescence generated by the activity of a predetermined enzyme bonded to the target substance or to a molecule specifically bonded thereto. The term “fluorescence generated by the activity of an enzyme” refers to, for example, fluorescence emitted by a fluorescent substance liberated as a result of the degradation of a fluorescent substrate by the activity of the enzyme. A method of detecting the fluorescence is, for example, a method including using a fluorescence microscope and an image sensor.

In addition, the detecting step preferably includes a step of detecting whether or not the detection particle is stored in each of the microwells. A method of detecting whether or not the detection particle is stored in each of the microwells is, for example, a method of observing the presence or absence of the detection particle under a microscope. In addition, a method of detecting the presence or absence of the detection particle is, for example, a method including detecting light scattered by the detection particle, or a method including utilizing potential measurement with a field-effect transistor. Further, any other detection method is, for example, a method including adding a standard fluorescent substance to the dispersion medium containing the detection particles to detect fluorescence as described above. Herein, a method of detecting the fluorescence is, for example, a method including using a fluorescence microscope or an image sensor as in the foregoing description.

When the fluorescence is detected by both of the method of detecting whether or not the detection particle that has captured the target substance is stored, and the method of detecting whether or not the detection particle is stored in each of the microwells, fluorescence wavelengths in the respective detections are preferably sufficiently distant from each other. Thus, the respective detections are facilitated.

The flow of the detecting step is described in more detail below.

For example, when the fluorescence emitted by the fluorescent substance liberated as a result of the degradation of the fluorescent substrate by the predetermined enzyme bonded to the target substance or to a molecule specifically bonded thereto is detected by incubation, first, a bright-field image and a fluorescence image are acquired after the incubation. A fluorescence microscope or the like may be used in the acquisition of the bright-field image and the fluorescence image.

In the acquired bright-field image, the detection particles stored in the microwells and the microwells are photographed. In other words, the bright-field image is taken for sensing the microwells and the detection particles. Herein, when a standard fluorescent substance or the like is loaded into a reaction liquid to cause all the microwells to emit fluorescence, a fluorescence image taken under photographing conditions commensurate with the fluorescence wavelength of the standard fluorescent substance can be acquired instead of the bright-field image. In addition, a fluorescence image for judging the positions of the detection particles may be taken by utilizing the autofluorescence of each of the detection particles.

Next, a masked image of the microwells (well: 1, portion except the well: 0) (hereinafter also referred to as “well-masked image”) and an image obtained by masking portions corresponding to the microwells having stored therein the detection particles (hereinafter also referred to as “particle-masked image”) are produced by using the bright-field image.

Finally, the fluorescence intensity of each of the microwells is calculated by using the fluorescence image, the well-masked image, and the particle-masked image, and the microwell, in which the detection particle is present and which has fluorescence intensity exceeding a predetermined threshold, is identified (positive judgment). The threshold may be automatically determined from the fluorescence intensity of each of the microwells by using Otsu's method or the like, or may be determined from a variation in fluorescence intensity measured by using a well plate in which no detection particles are stored. Alternatively, the threshold may be calculated by using information on the fluorescence intensity of each of the microwells in which no detection particles are stored.

As a result of the detecting step, the number of the microwells having stored therein the detection particles, and the number of the microwells having stored therein the particles that have each captured the target substance out of the detection particles can be elucidated. The ratio of the number of the detection particles that have each captured the target substance to the total number of the detection particles encapsulated in the microwells can be calculated by using those values. Thus, the concentration of the target substance can be determined.

In addition, when a fluorescent substance in the array is detected with a fluorescence microscope, an image sensor, or the like, in the case where the magnification of the microscope or the sensor is excessively high, an observation range narrows to reduce detection accuracy. Accordingly, an effective observation range is as follows: such a magnification that at least about 25,000 microwells can be observed is preferred, and the magnification at which 1,000,000 microwells can be observed is more preferred. When the observation range is wide, the plurality of samples and the plurality of items described above can be inspected at one time.

### EXAMPLES

The present invention is described in more detail below by way of Examples and Comparative Examples. The present invention is by no means limited to the following Examples, and various modifications may be made without departing from the gist of the present invention.

### (Production of Base Plate 1)

The base plate 11 illustrated in FIG. 1 was produced through a fluororesin-applying step, a photolithography step, and an etching-resist-removing step.

A quartz substrate (synthetic quartz substrate AQ grade, thickness: 1 mm, manufactured by AGC Inc.) was used as a substrate to be used in the production of the base plate 11. In the fluororesin-applying step, the above-mentioned substrate was treated with a silane coupling agent (KBE-903, manufactured by Shin-Etsu Silicone), and then a fluororesin (CYTOP (trademark) CTL-809A, manufactured by AGC Inc.) was applied thereto.

In the photolithography step, a positive photoresist (AZ P4903, AZ Electronic Materials) was applied to the quartz substrate. Next, the resultant was exposed to UV light from above through a photomask having a target pattern, and was subjected to development treatment with an alkali. The development treatment dissolved the photoresist only in the portion irradiated with the UV light to expose a hydrophobic resin layer.

In the etching-resist-removing step, part of the resin layer was removed by etching with oxygen plasma through the photoresist that had been partly dissolved. Thus, a hydrophobic partition wall was formed.

Finally, the photoresist was dissolved with an organic solvent. Thus, the target array 14 formed of the plurality of microwells 13 was formed. The diameter of the circular opening of each of the microwells 13 was 5 μm, the depth and volume of the microwell were 4 μm and 78.5 fL, respectively, a pitch between the microwells was 10 μm, and the number of the wells was about 1,000,000. A region on the base plate 11 where the array 14 was formed was a square about 10 mm on a side. Thus, a base plate 1 was produced.

### (Production of Wiping Member 1)

The wiping member 12 illustrated in FIG. 1 was produced by using a quartz substrate (synthetic quartz substrate AQ grade, thickness: 1 mm, manufactured by AGC Inc.) as a substrate serving as a material through waterjet processing. The member was defined as a wiping member 1. In addition, the partition wall member 15 illustrated in FIG. 1 was produced in the same manner as in the wiping member 1. The member was defined as a partition wall member 1.

### (Production of Wiping Member 2)

The wiping member 12 (partition wall member 15) illustrated in FIG. 4 was produced by using a quartz substrate (synthetic quartz substrate AQ grade, thickness: 1 mm, manufactured by AGC Inc.) as a substrate serving as a material through waterjet processing. The member was defined as a wiping member 2. That is, the wiping member 2 is a member that also functions as a partition wall member. The opening of the first region 16 and the opening of the second region 17 were each a square shape 11 mm on a side.

### (Production of Wiping Member 3)

The wiping member 12 (partition wall member 15) illustrated in FIG. 9 was produced by using a quartz substrate (synthetic quartz substrate AQ grade, thickness: 1 mm, manufactured by AGC Inc.) as a substrate serving as a material through waterjet processing. The member was defined as a wiping member 3. That is, the wiping member 3 is a member that also functions as a partition wall member. The width of each of the first partition 51 and the second partition 52 in the second direction B was 2 mm. In addition, the openings of the third regions 16a and 16b separated from each other by the first partition 51, and the openings of the fourth regions 17a and 17b separated from each other by the second partition 52 were each a rectangular shape whose short side and long side had lengths of 4.5 mm and 11 mm, respectively.

### (Production of Wiping Member 4)

A wiping member produced by depositing aluminum from the vapor onto one surface of the wiping member 2 (surface serving as the third surface 12c or the fourth surface 12d (see FIG. 6E)) was defined as a wiping member 4. That is, as in the wiping member 2, the wiping member 4 is a member that also functions as a partition wall member.

### (Production of Wiping Member 5)

In this example, the wiping member 12 (partition wall member 15) illustrated in FIG. 4 was produced as a wiping member 5 by using a transparent polystyrene plate having a thickness of 1 mm through waterjet processing. The opening of the first region 16 and the opening of the second region 17 in FIG. 4 were each a square shape 11 mm on a side.

### (Preparation of Detection Particles 1)

A dispersion of magnetic particles (Magnosphere (trademark) MS160/Carboxyl, manufactured by JSR Corporation) was prepared, and was defined as a dispersion of detection particles 1.

### (Preparation of Detection Particles 2)

A dispersion of magnetic particles (Magnosphere (trademark) MS300/Carboxyl, manufactured by JSR Corporation) was prepared, and was defined as a dispersion of detection particles 2.

### (Preparation of Detection Particles 3)

0.5 Gram of HIPRESICA (trademark) N3N (particle diameter: 2.9 μm) manufactured by Ube Exsymo Co., Ltd. was prepared as raw material particles, and the particles were dispersed in the mixed solution of 75 mL of ethanol (manufactured by Kishida Chemical Co., Ltd.) and 75 mL of pure water. Next, 1.5 mL of tetraethoxysilane (TEOS) (manufactured by Kishida Chemical Co., Ltd.) was added to the dispersion, and 22.5 mL of 28% ammonia water (manufactured by Kishida Chemical Co., Ltd.) was added as a catalyst to the mixture. The mixture was subjected to a reaction for 1.5 hours while being stirred. After the reaction, the solvent was removed by centrifugation, and the residue was washed with pure water seven times. Thus, silica layers were formed on the raw material particles.

The resultant silica-coated particles were dispersed in the mixed solution of mL of ethanol and 10 mL of pure water, and 100 μL of 3-methacryloxypropyltrimethoxysilane (LS-3380, manufactured by Shin-Etsu Chemical Co., Ltd.) was added as a silane coupling agent to the dispersion, followed by sufficient mixing. Next, 2 mL of 28% ammonia water was added to the mixture, and the whole was stirred for 1.5 hours. Next, the solvent was removed by centrifugation, and the residue was sufficiently washed with pure water. After that, 60 mL of pure water subjected to nitrogen bubbling was added to the washed product to provide an aqueous dispersion. Next, the aqueous dispersion was loaded into a four-necked flask (200 mL), and was subjected to nitrogen bubbling, followed by stirring at a stirring rate of 200 rpm for 15 minutes. Subsequently, the nitrogen bubbling was switched to a nitrogen flow, and then 50 μL of a styrene monomer (manufactured by Kishida Chemical Co., Ltd.) was added to the aqueous dispersion. Next, 0.02 g of potassium persulfate (manufactured by Sigma-Aldrich Co. LLC) was dissolved in 2 mL of pure water deaerated in advance by nitrogen bubbling, and 1 mL of the solution was added into the flask. Next, the mixture in the flask was heated with an oil bath at 35° C. for 30 minutes, and then the temperature was increased to 60° C., followed by the holding of the mixture for 1 hour. Subsequently, 200 μL of glycidyl methacrylate (manufactured by Kishida Chemical Co., Ltd.) was added to the mixture, and the whole was further held for 12 hours. Thus, polymerization was completed. After the completion of the polymerization, the solvent was removed by centrifugation, and the residue was sufficiently washed with pure water.

Subsequently, 10 mg of the resultant particles were dispersed in 5 mL of pure water. Separately from the particle dispersion, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added as a pH adjuster to the solution. 1 Milliliter of the solution was added to the particle dispersion, and the mixture was sufficiently stirred, followed by heating treatment at 60° C. for 3 hours. After that, the solvent was removed by centrifugation, and the residue was sufficiently washed with pure water. Thus, a dispersion of detection particles 3 was obtained.

The solid content concentration of the dispersion of the detection particles 3 determined by thermogravimetry-differential thermal analysis (TG-DTA) was 30 mg/mL. The diameter of each of the detection particles and the specific gravity of the particles were assumed to be 3.0 μm and 2.1 g/cm³, respectively, and the particle number concentration of the dispersion was set to 1.0×10⁹ particles/mL.

### (Production of Detection Particles 4)

The following composite particles were produced as detection particles 4: the composite of Cas12a and crRNA was bonded to each of the particles.

First, a dispersion of magnetic particles (Magnosphere (trademark) MS300/Carboxyl) was loaded into a microtube, and the magnetic particles were precipitated with a magnet. After the supernatant had been removed, a MES buffer solution (100 mM, pH: 5.4) was added to the magnetic particle pellet to redisperse the particles, and N-hydroxysulfosuccinimide (sulfo-NHS) and a water-soluble carbodiimide (WSC) were added to the dispersion. After that, the mixture was stirred at 25° C. for 1 hour, and the magnetic particles were recovered with a magnet.

Subsequently, the recovered magnetic particles were washed with a MES buffer solution, and were dispersed in the MES buffer solution, followed by the addition of an arbitrary amount of an anti-His-tag antibody (anti-His-tag mAb, MBL Life Science) thereto. After that, the mixture was stirred at 25° C. for 2 hours.

Subsequently, a large excess of ethanolamine was added to the mixture to deactivate active groups on the surfaces of the magnetic particles. The magnetic particles were recovered with a magnet, and the recovered magnetic particles were washed with a MES buffer solution. Thus, antibody-immobilized particles were produced.

A storage buffer (10 mM HEPES-NaOH (pH: 7.9), 50 mM KCl, 1 mM EDTA, and 10% glycerol) was added to the resultant antibody-immobilized particles to prepare an antibody-immobilized particle liquid. The antibody-immobilized particle liquid was stored at 4° C. until its use.

Next, Cas12a and crRNA that had been diluted were mixed at a concentration ratio (molar ratio) of 1:1.25, and the mixture was incubated at 37° C. for 30 minutes to produce a Cas12a-crRNA composite.

In addition, the produced antibody-immobilized particle liquid (1 wt %) was dispensed in a 2-milliliter sample tube (manufactured by VIOLAMO, model number: 1-1600-04). After the liquid had been stirred, the sample tube was stood on a magnetic stand (MAGICAL TRAPPER, manufactured by Toyobo Co., Ltd., model number: MGS-101), and was left to stand still for 1 minute. After that, the solvent was removed by removing the supernatant. Phosphate buffered saline containing 0.05% Tween 20 (PBS-T) was added as a particle-washing liquid to the residue, and the mixture was stirred. After that, the solvent was removed in the same manner as that described above. The foregoing operation was repeated twice to wash the particles.

The antibody-immobilized particles after the washing were suspended in PBS-T, and the Cas12a-crRNA solution prepared in the foregoing was added thereto so as to have an arbitrary concentration, followed by stirring. After that, the mixture was subjected to a reaction by using a shaker for 1 hour. Herein, the used Cas12a has a His-tag on an N-terminal thereof, and hence the Cas12a and each of the antibody-immobilized particles are bonded to each other through an antigen-antibody reaction between the His-tag of the Cas12a and the anti-His-tag antibody of the antibody-immobilized particle. Thus, the following detection particles 4 were produced: the composite of Cas12a and crRNA was bonded to each of the particles. The final particle number concentration of the detection particles 4 in their dispersion was calculated to be 3.1×10⁸ particles/mL.

After the reaction, the solvent was removed, and the residue was subjected to a washing operation with PBS-T. After the washing, the washed product was suspended in purified water, and the suspension was stirred, followed by its storage at 4° C. until its use.

### (Preparation of Dispersion Medium 1 Containing Detection Particles 1)

10 Microliters of the aqueous dispersion (10 mg/mL, 4.0×10⁹ particles/mL) of the detection particles 1 was diluted with 489.25 μL of a buffer (A), and 0.75 μL of 1 mM Alexa Fluor 647 was further added as a standard fluorescent substance to the diluted product to provide a dispersion medium 1 containing the detection particles 1.

The composition of the buffer (A) is as described below.

Composition of buffer (A):
50 mM Tris-Cl (pH: 7.6)
10 mM CaCl₂)
0.01% Brij (trademark) 35

### (Preparation of Dispersion Medium 2 Containing Detection Particles 2)

16.7 Microliters of the aqueous dispersion (10 mg/mL, 6.0×10⁸ particles/mL) of the detection particles 2 was diluted with 482.55 μL of a buffer (A), and 0.75 μL of 1 mM Alexa Fluor 647 was further added as a standard fluorescent substance to the diluted product to provide a dispersion medium 2 containing the detection particles 2.

### (Preparation of Dispersion Medium 3 Containing Detection Particles 3)

10 Microliters of the dispersion (30 mg/mL, 1.0×10⁹ particles/mL) of the detection particles 3 was diluted with 490 μL of a buffer (A) to provide a dispersion medium 3 containing the detection particles 3.

### (Preparation of Dispersion Medium 4 Containing Detection Particles 4)

First, synthetic DNA (hereinafter referred to as “DNA_113 bp”) was used as DNA serving as a target substance. The DNA_113 bp was diluted with purified water to prepare a DNA stock solution (4 nM), and its concentration was determined through measurement with Qubit (trademark) 2.0 Fluorometer (manufactured by Life Technologies Corporation). The DNA stock solution (4 nM) was diluted with purified water to prepare a DNA solution (0.684 nM). Further, the DNA solution (0.684 nM) was diluted with purified water to prepare a DNA solution 1 (0.228 nM).

The base sequence (SEQ ID NO: 1) of the DNA_113 bp is as described below. ctcacgccttatgactgcccttatgtcaccgcttatgtctcccgatatcacacccgttatctcagccctaatctctgcggtttagtct ggccttaatccatgcctcatagcta

Next, 7.3 μL of the aqueous dispersion (3.1×10⁸ particles/mL) of the detection particles 4, 22.7 μL of water, and 30 μL of the DNA solution 1 (0.228 nM) were mixed. The resultant mixed solution was subjected to a reaction at 37° C. for 30 minutes to form the composite of composite particles and the DNA.

Next, the following materials were prepared.
Reporter molecule solution (12 μM) containing 800 nM HiLyte 488: 25 μL
Tween 20 (5%): 10 μL
BSA (30%): 1 μL
Aqueous solution of spermine (50 mM): 4 μL
10× binding buffer: 10 μL

Those materials were mixed in a 1.5-milliliter microtube in advance. 50 Microliters of the above-mentioned composite of the composite particles and the DNA was added to the mixed solution to provide a dispersion medium 4 containing the detection particles 4.

A reporter molecule in a commercial kit (product name: DNaseAlert (trademark) Substrate Nuclease Detection System 11-02-01-04, manufactured by Integrated DNA Technologies, Inc.) was used as the reporter molecule solution. The reporter molecule includes HEX serving as a fluorescent substance and a quenching agent.

### (Preparation of Dispersion Medium 5 Containing Detection Particles 4)

First, DNA_113 bp was used as DNA serving as a target substance. The DNA_113 bp was diluted with purified water to prepare a DNA stock solution (4 nM), and its concentration was determined through measurement with Qubit 2.0 Fluorometer (manufactured by Life Technologies Corporation). The DNA stock solution (4 nM) was diluted with purified water to prepare a DNA solution (0.684 nM). Further, the DNA solution (0.684 nM) was diluted with purified water to prepare a DNA solution 2 (0.114 nM).

Next, 7.3 μL of the dispersion (3.1×10⁸ particles/mL) of the detection particles 4, 22.7 μL of water, and 30 μL of the DNA solution 2 (0.114 nM) were mixed. The resultant mixed solution was subjected to a reaction at 37° C. for 30 minutes to form the composite of composite particles and the DNA.

Next, the following materials were prepared.
Reporter molecule solution (12 μM) containing 800 nM HiLyte 488: 25 μL
Tween 20 (5%): 10 μL
BSA (30%): 1 μL
Aqueous solution of spermine (50 mM): 4 μL
10× binding buffer: 10 μL

Those materials were mixed in a 1.5-milliliter microtube in advance. 50 Microliters of the above-mentioned composite of the composite particles and the DNA was added to the mixed solution to provide a dispersion medium 5 containing the detection particles 4.

### Example 1

The combination of the base plate 1 and the wiping member 2 was selected as a microwell device, and the dispersion medium 2 containing the detection particles 2 was selected as a dispersion medium containing detection particles to be introduced into the array of the plate.

First, the base plate and the wiping member were set so that an arrangement illustrated in FIG. 6A was achieved. Next, 100 μL of the dispersion medium containing the detection particles was loaded into the first region of the wiping member with a micropipette. Immediately after the loading, the wiping member was slid on the base plate to a position illustrated in FIG. 6C with respect to the base plate, and then deaeration was performed by leaving the microwell device in a vacuum desiccator at 0.1 atm for about 30 seconds. After the deaeration, the device was left to stand still for 5 minutes, and the packing of the detection particles 2 into its microwells was awaited. During the still standing, 100 μL of SR-X Sealing Oil serving as a fluorine-based oil was loaded into the second region of the wiping member with a micropipette. Next, the wiping member was slowly slid on the base plate to a position illustrated in FIG. 6F with respect to the base plate to seal the microwells with the fluorine-based oil.

### Example 2

This Example was performed in the same manner as in Example 1 except that in Example 1, the sealing step was changed as described below.

While the microwell device was left to stand still in an arrangement illustrated in FIG. 6C, 100 μL of SR-X Sealing Oil serving as a fluorine-based oil was loaded into the second region of the wiping member with a micropipette. Next, the wiping member was slowly slid on the base plate to the position illustrated in FIG. 6F with respect to the base plate. Further, the wiping member was slid on the base plate to a position illustrated in FIG. 6G with respect to the base plate slowly and while the pressing of the wiping member against the base plate was loosened, to thereby seal the microwells with a thin layer of the oil and the wiping member.

### Example 3

In Example 2, the “dispersion medium 2 containing the detection particles 2” was changed to the “dispersion medium 3 containing the detection particles 3.” This Example was performed in the same manner as in Example 2 except the foregoing.

### Example 4

In Example 2, the “dispersion medium 2 containing the detection particles 2” was changed to the “dispersion medium 1 containing the detection particles 1.” This Example was performed in the same manner as in Example 2 except the foregoing.

### Example 5

In Example 2, the wiping member 2 was changed to the wiping member 4. The wiping member 4 was used while being set so that its metal-deposited surface was brought into contact with the surface of the base plate including the array. That is, a configuration in which the third surface of the wiping member 4 had the metal-deposited surface was adopted. This Example was performed in the same manner as in Example 2 except the foregoing.

### Example 6

In Example 5, the wiping member 4 was used while being set so that its surface opposite to the metal-deposited surface was brought into contact with the surface of the base plate including the array. That is, a configuration in which the fourth surface of the wiping member 4 had the metal-deposited surface was adopted. This Example was performed in the same manner as in Example 5 except the foregoing.

### Example 7

This Example was performed in the same manner as in Example 2 except that in Example 2, the introducing step was changed as described below.

The base plate and the wiping member were set so that the arrangement illustrated in FIG. 6C was achieved. Next, 100 μL of the dispersion medium 2 containing the detection particles 2 was loaded into the first region of the wiping member with a micropipette.

### Example 8

This Example was performed in the same manner as in Example 7 except that in Example 7, the “dispersion medium 2 containing the detection particles 2” was changed to the “dispersion medium 1 containing the detection particles 1,” and the packing step was changed as described below.

In the arrangement illustrated in FIG. 6C, deaeration was performed by leaving the microwell device in a vacuum desiccator at 0.1 atm for about 30 seconds. After the deaeration, the entirety of the array was traced from the back side of the base plate with a neodymium magnet (diameter: 15 mm, height: 10 mm, manufactured by Niroku Seisakusho) over 5 seconds. After that, the device was left to stand still for 5 minutes, and the packing of the detection particles 1 into its microwells was awaited.

### Example 9

The combination of the base plate 1 and the wiping member 3 was selected as a microwell device. In addition, the following two kinds were selected as dispersion media containing detection particles: the dispersion medium 1 containing the detection particles 1, and the dispersion medium 2 containing the detection particles 2.

First, the base plate and the wiping member were set so that the arrangement illustrated in FIG. 6C was achieved. Next, 80 μL of the dispersion medium 1 containing the detection particles 1 was loaded into one of the third region 16a, and 80 μL of the dispersion medium 2 was similarly loaded into the other third region 16b.

Subsequently, deaeration was performed by leaving the microwell device in a vacuum desiccator at 0.1 atm for about 30 seconds. After the deaeration, the device was left to stand still for 10 minutes, and the packing of the detection particles 1 and the detection particles 2 into its microwells was awaited. While the microwell device was left to stand still, 100 μL of SR-X Sealing Oil serving as a fluorine-based oil was loaded into each of the fourth regions 17a and 17b with a micropipette. Next, the wiping member was slowly slid on the base plate to the position illustrated in FIG. 6F with respect to the base plate. Further, the wiping member was slid on the base plate to the position illustrated in FIG. 6G with respect to the base plate slowly and while the pressing of the wiping member against the base plate was loosened, to thereby seal the microwells with a thin layer of the oil and the wiping member.

### Example 10

This Example was performed in the same manner as in Example 1 except that in Example 1, the sealing step was changed as described below.

After the packing step had been performed in the arrangement illustrated in FIG. 6C, the wiping member was slowly slid on the base plate to the position illustrated in FIG. 6F with respect to the base plate. After that, 100 μL of SR-X Sealing Oil serving as a fluorine-based oil was loaded into the second region of the wiping member with a micropipette along a wall of the hole. Thus, the microwells were sealed with the fluorine-based oil.

### Example 11

This Example was performed in the same manner as in Example 10 except that in Example 10, the introducing step was changed as described below.

First, the base plate and the wiping member were set so that the arrangement illustrated in FIG. 6C was achieved. Next, 100 μL of the dispersion medium 2 was loaded into the first region of the wiping member with a micropipette.

### Example 12

The combination of the base plate 1, the wiping member 1, and the partition wall member 1 was selected as a microwell device, and the dispersion medium 2 containing the detection particles 2 was selected as a dispersion medium containing detection particles to be introduced into the array of the plate.

First, the base plate, the wiping member, and the partition wall member were set so that an arrangement illustrated in FIG. 3A was achieved. Next, 100 μL of the dispersion medium 2 containing the detection particles 2 was loaded into a region on the base plate between the array and the first surface of the wiping member with a micropipette. Immediately after the loading, the wiping member was slid on the base plate to a position illustrated in FIG. 3C with respect to the base plate. After that, deaeration was performed by leaving the microwell device in a vacuum desiccator at 0.1 atm for about 30 seconds. After the deaeration, the device was left to stand still for 5 minutes, and the packing of the detection particles 2 into its microwells was awaited. After the still standing, the wiping member was slowly slid on the base plate to a position illustrated in FIG. 3E with respect to the base plate. After that, 100 μL of SR-X Sealing Oil serving as a fluorine-based oil was loaded into a region on the base plate between the array and the second surface of the wiping member with a micropipette. Immediately after the loading, the wiping member was slowly slid on the base plate to a position illustrated in FIG. 3F with respect to the base plate to seal the microwells with the oil.

### Example 13

This Example was performed in the same manner as in Example 12 except that in Example 12, the introducing step was changed as described below.

First, the base plate, the wiping member, and the partition wall member were set so that an arrangement illustrated in FIG. 3C was achieved. Next, 100 μL of the dispersion medium 2 containing the detection particles 2 was loaded onto the array with a micropipette along the first surface of the wiping member.

### Example 14

This Example was performed in the same manner as in Example 12 except that in Example 12, the sealing step was changed as described below.

After the packing step had been performed in the arrangement illustrated in FIG. 3C, the wiping member was slowly slid on the base plate to the position illustrated in FIG. 3E with respect to the base plate, and was then subsequently slid to the position illustrated in FIG. 3F. After that, 100 μL of SR-X Sealing Oil serving as a fluorine-based oil was loaded onto the array with a micropipette along the second surface of the wiping member. Thus, the microwells of the array were sealed with the oil.

### Example 15

This Example was performed in the same manner as in Example 13 except that in Example 13, the sealing step was changed as described below.

After the packing step had been performed in the arrangement illustrated in FIG. 3C, the wiping member was slowly slid on the base plate to the position illustrated in FIG. 3E with respect to the base plate, and was then subsequently slid to the position illustrated in FIG. 3F. After that, 100 μL of SR-X Sealing Oil serving as a fluorine-based oil was loaded onto the array with a micropipette along the second surface of the wiping member. Thus, the microwells of the array were sealed with the oil.

### Example 16

In Example 1, the “dispersion medium 2 containing the detection particles 2” was changed to the “dispersion medium 4 containing the detection particles 4.” This Example was performed in the same manner as in Example 1 except the foregoing.

### Example 17

In Example 2, the “dispersion medium 2 containing the detection particles 2” was changed to the “dispersion medium 4 containing the detection particles 4.” This Example was performed in the same manner as in Example 2 except the foregoing.

### Example 18

In Example 7, the “dispersion medium 2 containing the detection particles 2” was changed to the “dispersion medium 4 containing the detection particles 4.” This Example was performed in the same manner as in Example 7 except the foregoing.

### Example 19

In Example 8, the “dispersion medium 2 containing the detection particles 2” was changed to the “dispersion medium 4 containing the detection particles 4.” This Example was performed in the same manner as in Example 8 except the foregoing.

### Example 20

In Example 5, the “dispersion medium 2 containing the detection particles 2” was changed to the “dispersion medium 4 containing the detection particles 4.” This Example was performed in the same manner as in Example 5 except the foregoing.

### Example 21

In Example 6, the “dispersion medium 2 containing the detection particles 2” was changed to the “dispersion medium 4 containing the detection particles 4.” This Example was performed in the same manner as in Example 6 except the foregoing.

### Example 22

In Example 9, the two kinds, that is, the dispersion medium 1 containing the detection particles 1, and the dispersion medium 2 containing the detection particles 2 were changed to the following two kinds: the dispersion medium 4 containing the detection particles 4, and the dispersion medium 5 containing the detection particles 4. In addition, in the introducing step, the dispersion medium 4 containing the detection particles 4 was loaded into the one third region 16a, and the dispersion medium 5 containing the detection particles 4 was loaded into the other third region 16b. This Example was performed in the same manner as in Example 9 except the foregoing.

### Example 23

This Example was performed in the same manner as in Example 10 except that: first, 120 μL of SR-X Sealing Oil serving as a fluorine-based oil was applied to the entirety of the back surface of the wiping member with a micropipette; and then the oil-applied surface was brought into contact with the base plate, followed by the setting of the plate and the member so that the arrangement illustrated in FIG. 6A was achieved.

### Example 24

This Example was performed in the same manner as in Example 23 except that the wiping member 5 was selected instead of the wiping member 2.

### Example 25

This Example was performed in the same manner as in Example 24 except that pure water was used as a medium to be applied to the entirety of the back surface of the wiping member instead of the SR-X Sealing Oil serving as a fluorine-based oil.

### Example 26

This Example was performed in the same manner as in Example 24 except that the operation of deaerating the microwell device after the first sliding operation was not performed.

### Example 27

This Example was performed in the same manner as in Example 26 except that the wiping member was set after the base plate had been arranged so as to be brought into contact with the magnet surface of a magnet soft case (Crown, CR-MGA5).

### Example 28

This Example was performed in the same manner as in Example 27 except that: a magnet bar (KOKUYO Co., Ltd., MAKU-201NB) was used instead of the magnet soft case; and the wiping member was set after the base plate had been arranged so as to be brought into contact with the magnet surface of the bar.

### Example 29

This Example was performed in the same manner as in Example 28 except that: after the packing of the detection particles 2 into the microwells in the arrangement illustrated in FIG. 6C had been awaited for 5 minutes, the microwell device was lowered from the magnet bar, and a state in which no magnetic field was applied was held for 1 minute; and then the wiping member was slowly slid on the base plate to the position illustrated in FIG. 6F.

### Comparative Example 1

The combination of the base plate 1 and the wiping member 2 was selected as a microwell device, and the dispersion medium 2 containing the detection particles 2 was selected as a dispersion medium containing detection particles to be introduced into the array of the plate.

First, the base plate and the wiping member were set so that the arrangement illustrated in FIG. 6A was achieved. Next, 100 μL of the dispersion medium 2 containing the detection particles 2 was loaded into the first region of the wiping member with a micropipette. Immediately after the loading, the wiping member was slid on the base plate to the position illustrated in FIG. 6C with respect to the base plate, and then deaeration was performed by leaving the microwell device in a vacuum desiccator at 0.1 atm for about 30 seconds. After the deaeration, the device was left to stand still for 5 minutes, and the packing of the detection particles 2 into its microwells was awaited. After the still standing, the dispersion medium 2 in the first region was slowly sucked up with a micropipette by one sucking operation. After that, 100 μL of SR-X Sealing Oil serving as a fluorine-based oil was loaded into the first region with a micropipette. Thus, the microwells were sealed with the oil.

### Comparative Example 2

This Example was performed in the same manner as in Comparative Example 1 except that in Comparative Example 1, the introducing step was changed as described below.

First, the base plate and the wiping member were set so that the arrangement illustrated in FIG. 6C was achieved. Next, 100 μL of the dispersion medium 2 was loaded into the first region of the wiping member with a micropipette.

### Comparative Example 3

Simoa Discs (16 discs): 100001 (manufactured by Quanterix Corp.) were each used as a microwell device. According to specifications, the opening diameter, depth, and volume of each of microwells are 4.25 μm, 3.25 μm, and 50 fL, respectively, the total number of the microwells is 239,000, and a region on a base plate where an array is formed is a rectangle whose short side and long side have lengths of 3 mm and 4 mm, respectively. The dispersion medium 2 containing the detection particles 2 was selected as a dispersion medium containing detection particles to be introduced into the array.

25 Microliters of the dispersion medium 2 containing the detection particles 2 was slowly loaded into the inlet hole of the microwell device with a micropipette. After the loading, deaeration was performed by leaving the microwell device in a vacuum desiccator at 0.1 atm for about 30 seconds. After the deaeration, the device was left to stand still for 5 minutes, and the packing of the detection particles 2 into its microwells was awaited. After the still standing, 100 μL of SR-X Sealing Oil serving as a fluorine-based oil was loaded into the inlet hole of the microwell device with a micropipette. Thus, the microwells were sealed with the oil. When the fluorine-based oil for sealing was loaded from the inlet hole, a small amount of the dispersion medium 2 was pushed out of the outlet hole of the device.

### Comparative Example 4

This Comparative Example was performed in the same manner as in Comparative Example 3 except that in Comparative Example 3, the dispersion medium 1 containing the detection particles 1 was selected instead of the dispersion medium 2 containing the detection particles 2, and the still standing time was set to 10 minutes. When the fluorine-based oil for sealing was loaded from the inlet hole of the microwell device, a small amount of the dispersion medium 1 was pushed out of the outlet hole thereof as in Comparative Example 3.

### Comparative Example 5

This Comparative Example was performed under the following conditions so that PITAT METHOD serving as a particle encapsulation method described in H. Yaginuma et al., Lab on a Chip, 2022, 22, 2001-2010 was reproduced.

The base plate 1 was selected as a microwell device, and the dispersion medium 2 containing the detection particles 2 was selected as a dispersion medium containing detection particles to be introduced into the array of the plate.

100 Microliters of the dispersion medium 2 containing the detection particles 2 was loaded onto the array of the base plate with a micropipette.

The microwell device was deaerated by being left to stand still on an aluminum block, which had been cooled with ice, for 1 minute. After that, the temperature of the device was returned to a room temperature of 25° C., and the device was left to stand still for 5 minutes.

After the still standing, one SCOTCH (trademark) 145RN tape (manufactured 3M Company) cut out into a rectangular shape whose short side and long side had lengths of about 6 mm and about 15 mm, respectively was lightly bonded so as to traverse the array. After that, a plastic rod around which a Kapton tape had been wound so as to have a thickness of about 0.2 mm was rolled so that its portion having wound therearound the Kapton tape was brought into abutment with the temporarily fixed 145RN tape. Thus, the 145RN tape was firmly bonded to the array, and hence the microwells of the array were sealed with the 145RN tape.

When the microwells were sealed with the 145RN tape, the dispersion medium 2 was pushed out of a side of the tape. Further, when the plastic rod was rolled, the dispersion medium 2 that had been pushed out adhered to the Kapton tape portion wound around the rod, and the dispersion medium 2 also adhered to the surface of the 145RN tape sealing the microwells.

### [Evaluation]

The results of Examples were evaluated in terms of the following three items: a packing ratio, the width of an effective observation range, and the staining of a microwell device.

With regard to actual detection sensitivity, first, how many detection particles are packed into microwells is important. Even when the detection particles have each captured a target substance in pretreatment, the substance cannot be detected unless the detection particles are packed into the microwells. Accordingly, the pretreatment is unsuitable as a particle encapsulation method.

Next, the width of the effective observation range is important. Even when the particles are packed into the microwells, at the time of actual detection of the target substance, an aggregate of the detection particles is, or air bubbles are, present on the microwells, or a sealing medium cannot uniformly cover a microwell array in some cases. A method that may cause such cases is unsuitable as a particle encapsulation method because a region where the positions of the microwells themselves, the detection particles, and light-emitting microwells can be detected in predetermined image processing narrows.

In addition, in an actual handling operation, the microwell device itself is stained with the dispersion medium or the sealing medium discharged in the sealing step or the like, and an inspection apparatus is also stained in the subsequent detecting step or the like in some cases. A method that may cause such cases may affect the accuracy of inspection results. Accordingly, even when the target substance can be detected with high sensitivity, the method is unsuitable as a particle encapsulation method.

In each of Examples in which the dispersion medium 3 containing the detection particles 3 and the dispersion medium 4 containing the detection particles 4 were selected as dispersion media containing detection particles to be introduced into arrays out of Examples described above, the generation of fluorescence was accelerated by subjecting the detection particles to a reaction through incubation at 37° C.

The packing ratio and the width of the effective observation range were evaluated with a fluorescence microscope (BZ-X800 (manufactured by Keyence Corporation)) under such a range condition that about 25,000 microwells were able to be observed.

The packing ratio of the detection particles was specifically determined as described below. First, the positions of the microwells were identified based on the fluorescence of a standard fluorescent substance, and then the microwells having in themselves contour edges resulting from the detection particles were sampled by image processing. After that, the packing ratio of the detection particles was determined from a ratio between the total number of the microwells and the number of the microwells having packed thereinto the detection particles in an observation range. When the dispersion medium was free of the standard fluorescent substance, the positions of the microwells were identified based on a masked image of the microwells acquired by packing a solvent containing the same standard fluorescent substance into the microwells in advance. Subsequently, the microwells having in themselves contour edges resulting from the detection particles were sampled by image processing, and the packing ratio of the detection particles was determined from a ratio between the total number of the microwells and the number of the microwells having packed thereinto the detection particles in an observation range.

In addition, the fluorescence intensity of each of the microwells was measured, and the microwell whose fluorescence intensity exceeded a predetermined threshold was judged to be a positive microwell in which fluorescence was caused by the enzyme activity of the target substance, followed by the measurement of the number of the positive microwells. Then, a positive ratio was calculated as a reference value from a ratio between the number of the microwells having packed thereinto the detection particles and the number of the positive microwells.

The packing ratio is shown in Table 1, and the positive ratio is shown in Table 2.

Measurement conditions for the respective fluorescent substances are as described below.
(Dispersion Medium 1 containing Detection particles 1 and Dispersion Medium 2 containing Detection particles 2)
Standard fluorescent substance (Alexa Fluor (trademark) 647): ex 650 nm, em 670 nm
(Dispersion Medium 4 containing Detection particles 4 and Dispersion Medium 5 containing Detection particles 4)
HEX: ex 533 nm, em 559 nm
Standard fluorescent substance (HiLyte (trademark) Fluor 488): ex 499 nm, em 523 nm

TABLE 1

| | Detection | Dispersion | Packing |
|---|---|---|---|
| Example | particles | medium | ratio |
| Example 1 | Detection | Dispersion | 55% |
| | particles 2 | medium 2 | |
| Example 2 | Detection | Dispersion | 57% |
| | particles 2 | medium 2 | |
| Example 3 | Detection | Dispersion | 34% |
| | particles 3 | medium 3 | |
| Example 4 | Detection | Dispersion | 72% |
| | particles 1 | medium 1 | |
| Example 5 | Detection | Dispersion | 56% |
| | particles 2 | medium 2 | |
| Example 6 | Detection | Dispersion | 57% |
| | particles 2 | medium 2 | |
| Example 7 | Detection | Dispersion | 58% |
| | particles 2 | medium 2 | |
| Example 8 | Detection | Dispersion | 85% |
| | particles 1 | medium 1 | |
| Example 9 | Detection | Dispersion | 67% |
| | particles 1 | medium 1 | |
| | Detection | Dispersion | 55% |
| | particles 2 | medium 2 | |
| Example 10 | Detection | Dispersion | 57% |
| | particles 2 | medium 2 | |
| Example 11 | Detection | Dispersion | 55% |
| | particles 2 | medium 2 | |
| Example 12 | Detection | Dispersion | 53% |
| | particles 2 | medium 2 | |
| Example 13 | Detection | Dispersion | 51% |
| | particles 2 | medium 2 | |
| Example 14 | Detection | Dispersion | 52% |
| | particles 2 | medium 2 | |
| Example 15 | Detection | Dispersion | 53% |
| | particles 2 | medium 2 | |
| Example 16 | Detection | Dispersion | 49% |
| | particles 4 | medium 4 | |
| Example 17 | Detection | Dispersion | 45% |
| | particles 4 | medium 4 | |
| Example 18 | Detection | Dispersion | 46% |
| | particles 4 | medium 4 | |
| Example 19 | Detection | Dispersion | 61% |
| | particles 4 | medium 4 | |
| Example 20 | Detection | Dispersion | 41% |
| | particles 4 | medium 4 | |
| Example 21 | Detection | Dispersion | 40% |
| | particles 4 | medium 4 | |
| Example 22 | Detection | Dispersion | 47% |
| | particles 4 | medium 4 | |
| | Detection | Dispersion | 45% |
| | particles 4 | medium 5 | |
| Example 23 | Detection | Dispersion | 56% |
| | particles 2 | medium 2 | |
| Example 24 | Detection | Dispersion | 55% |
| | particles 2 | medium 2 | |
| Example 25 | Detection | Dispersion | 54% |
| | particles 2 | medium 2 | |
| Example 26 | Detection | Dispersion | 50% |
| | particles 2 | medium 2 | |
| Example 27 | Detection | Dispersion | 59% |
| | particles 2 | medium 2 | |
| Example 28 | Detection | Dispersion | 60% |
| | particles 2 | medium 2 | |
| Example 29 | Detection | Dispersion | 62% |
| | particles 2 | medium 2 | |
| Comparative | Detection | Dispersion | 44% |
| Example 1 | particles 2 | medium 2 | |
| Comparative | Detection | Dispersion | 43% |
| Example 2 | particles 2 | medium 2 | |
| Comparative | Detection | Dispersion | 21% |
| Example 3 | particles 2 | medium 2 | |
| Comparative | Detection | Dispersion | 46% |
| Example 4 | particles 1 | medium 1 | |
| Comparative | Detection | Dispersion | 41% |
| Example 5 | particles 2 | medium 2 | |

TABLE 2

| | Detection | Dispersion | Positive |
|---|---|---|---|
| Example | particles | medium | ratio |
| Example 16 | Detection | Dispersion | 100% |
| | particles 4 | medium 4 | |
| Example 17 | Detection | Dispersion | 100% |
| | particles 4 | medium 4 | |
| Example 18 | Detection | Dispersion | 100% |
| | particles 4 | medium 4 | |
| Example 19 | Detection | Dispersion | 100% |
| | particles 4 | medium 4 | |
| Example 20 | Detection | Dispersion | 100% |
| | particles 4 | medium 4 | |
| Example 21 | Detection | Dispersion | 100% |
| | particles 4 | medium 4 | |
| Example 22 | Detection | Dispersion | 100% |
| | particles 4 | medium 4 | |
| | Detection | Dispersion | 100% |
| | particles 4 | medium 5 | |

In addition, the staining of the microwell device was evaluated as described below.

After the sealing step, the microwell device was left to stand still at an angle of 5° for 10 minutes, and whether or not concern was raised in that the dispersion medium or the sealing medium introduced into the microwell device moved along the surface of the microwell device to stain the microwell device was observed. The angle of 5° was set as the tilt of the microwell device assumed in the typical handling operation of the microwell device.

In the present invention, the respective evaluations of the packing ratio, the width of the effective observation range, and the staining of the device were ranked based on the following evaluation criteria. In the present invention, levels A and B were defined as preferred levels, and a level C was defined as an unacceptable level. The respective evaluation results are collectively shown in Table 3.

### <Packing Ratio>

A: The packing ratio is more than 50%.
B: The packing ratio is more than 30% and 50% or less.
C: The packing ratio is 30% or less.

### <Width of Effective Observation Range>

A: The ratio of the number of the microwells in a region where light-emitting microwells cannot be correctly detected by image processing to the total number of the microwells in the observation range is less than 5% at the maximum.
B: The ratio of the number of the microwells in a region where light-emitting microwells cannot be correctly detected by image processing to the total number of the microwells in the observation range is 5% or more and less than 20% at the maximum.
C: The ratio of the number of the microwells in a region where light-emitting microwells cannot be correctly detected by image processing to the total number of the microwells in the observation range is 20% or more at the maximum.

### <Staining of Device>

A: Even when the microwell device is held for 10 minutes while being tilted at an angle of 5°, no concern is raised about the staining of the microwell device.
C: When the microwell device is held for 10 minutes while being tilted at an angle of 5°, concern is raised about the staining of the microwell device.

TABLE 3

| | Evaluation result | | |
|---|---|---|---|
| | Packing | Width of effective | Staining of |
| Example | ratio | observation range | device |
| Example 1 | A | A | A |
| Example 2 | A | A | A |
| Example 3 | B | A | A |
| Example 4 | A | A | A |
| Example 5 | A | A | A |
| Example 6 | A | A | A |
| Example 7 | A | A | A |
| Example 8 | A | A | A |
| Example 9 | A/A | A | A |
| Example 10 | B | A | A |
| Example 11 | A | A | A |
| Example 12 | A | B | A |
| Example 13 | A | B | A |
| Example 14 | A | B | A |
| Example 15 | A | B | A |
| Example 16 | B | A | A |
| Example 17 | B | A | A |
| Example 18 | B | A | A |
| Example 19 | A | A | A |
| Example 20 | B | A | A |
| Example 21 | B | A | A |
| Example 22 | B/B | A | A |
| Example 23 | A | A | A |
| Example 24 | A | A | A |
| Example 25 | A | A | A |
| Example 26 | A | A | A |
| Example 27 | A | A | A |
| Example 28 | A | A | A |
| Example 29 | A | A | A |
| Comparative | B | C | A |
| Example 1 | | | |
| Comparative | B | C | A |
| Example 2 | | | |
| Comparative | C | C | C |
| Example 3 | | | |
| Comparative | B | C | C |
| Example 4 | | | |
| Comparative | B | C | C |
| Example 5 | | | |

According to the present invention, there can be provided a simple particle encapsulation method by which particles can be uniformly encapsulated in microwells.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2024-042796, filed Mar. 18, 2024, and Japanese Patent Application No. 2025-018577, filed Feb. 6, 2025, which are hereby incorporated by reference herein in their entirety.

What is claimed is:

1. A particle encapsulation method comprising:
a preparing step of preparing a microwell device including: a base plate having arranged in an upper surface thereof an array formed of a plurality of microwells; and a wiping member;
an introducing step of introducing a dispersion medium containing detection particles into the array;
a packing step of packing the detection particles into the microwells;
a removing step of removing the dispersion medium present outside the microwells; and
a sealing step of sealing the microwells with a sealing medium,
wherein the removing step includes sliding the wiping member in a first direction under a state in which the wiping member is brought into contact with the upper surface of the base plate, to thereby move the dispersion medium present outside the microwells to an outside of the array.

2. The particle encapsulation method according to claim 1,
wherein the microwell device further includes a partition wall member,
wherein the partition wall member includes two first portions, which extend in the first direction in an outer side of the array of the upper surface of the base plate, across the array, and the partition wall member further includes at least one second portion, which connects corresponding end portions of the two first portions to each other and extends in a second direction perpendicular to the first direction, and
wherein the wiping member is configured to be used under a state of being arranged between the two first portions so as to be capable of sandwiching the array together with the second portion.

3. The particle encapsulation method according to claim 2,
wherein the wiping member includes a first surface that is brought into abutment with the dispersion medium to move the dispersion medium in the removing step, and
wherein the introducing step includes introducing the dispersion medium containing the detection particles into a region sandwiched between the first surface and the second portion.

4. The particle encapsulation method according to claim 3, wherein the wiping member is configured to be used under a state of being connected to each of the two first portions.

5. The particle encapsulation method according to claim 2,
wherein the wiping member includes a second surface that is brought into abutment with the sealing medium to move the sealing medium in the sealing step, and
wherein the sealing step includes introducing the sealing medium into a region sandwiched between the second surface and the second portion.

6. The particle encapsulation method according to claim 5, wherein the wiping member is configured to be used under a state of being connected to each of the two first portions.

7. The particle encapsulation method according to claim 1,
wherein the wiping member includes a first surface that is brought into abutment with the dispersion medium to move the dispersion medium in the removing step, and one or more first partitions configured to extend from the first surface in the first direction to inhibit movement of the dispersion medium toward a second direction perpendicular to the first direction, and
wherein the introducing step includes introducing the dispersion medium containing the detection particles into regions defined by the first partitions and the first surface.

8. The particle encapsulation method according to claim 7,
wherein the wiping member includes a second surface that is brought into abutment with the sealing medium to move the sealing medium in the sealing step, and the wiping member includes one or more second partitions configured to extend from the second surface in the first direction to inhibit movement of the sealing medium toward the second direction,
wherein the second partitions are arranged at corresponding positions as those of the first partitions in the second direction, and
wherein the sealing step includes introducing the sealing medium into regions defined by the second partitions and the second surface.

9. The particle encapsulation method according to claim 1,
wherein the base plate is formed of a material having translucency,
wherein a third surface of the wiping member to be brought into contact with the base plate includes a sealing surface having a size enough to cover the array, and the sealing surface is coated with a metal, and
wherein the sealing step includes covering the array with the sealing surface.

10. The particle encapsulation method according to claim 1,
wherein the base plate and the wiping member are each formed of a material having translucency,
wherein a third surface of the wiping member to be brought into contact with the base plate includes a sealing surface having a size enough to cover the array,
wherein a fourth surface opposite to the third surface of the wiping member to be brought into contact with the base plate includes a metal-coated surface that faces the sealing surface and has a size enough to cover the sealing surface, and
wherein the sealing step includes covering the array with the sealing surface.

11. The particle encapsulation method according to claim 1, wherein the sealing step includes sliding the wiping member in a direction opposite to the first direction under a state in which the wiping member is brought into contact with the upper surface of the base plate, to thereby move the sealing medium onto the microwells.

12. A microwell device comprising:
a base plate having arranged in an upper surface thereof an array formed of a plurality of microwells; and
a wiping member,
wherein the microwells are wells for storing detection particles dispersed in a dispersion medium, and
wherein the wiping member is configured to be capable of moving the dispersion medium present outside the microwells to an outside of the array by being slid in a first direction under a state of being brought into contact with the upper surface of the base plate.

13. The microwell device according to claim 12, further comprising a partition wall member,
wherein the partition wall member is configured to include at least a first portion, which extends in the first direction in an outer side of the array of the upper surface of the base plate, to inhibit a flow of the dispersion medium toward a second direction perpendicular to the first direction in the upper surface of the base plate.

14. The microwell device according to claim 13,
wherein the partition wall member includes: the two first portions arranged across the array; and at least one second portion, which connects corresponding end portions of the two first portions to each other and extends in the second direction, and
wherein the wiping member is configured to be used under a state of being arranged between the two first portions so as to be capable of sandwiching the array together with the second portion.

15. The microwell device according to claim 14, wherein the partition wall member includes the following two second portions: the second portion connecting the end portions of the two first portions on one side to each other; and the second portion connecting the end portions of the two first portions on another side to each other.

16. The microwell device according to claim 14, wherein the wiping member is configured to be used under a state of being connected to each of the two first portions.

17. The microwell device according to claim 12, wherein the wiping member includes a first surface configured to be brought into abutment with the dispersion medium to move the dispersion medium, and one or more first partitions configured to extend from the first surface in the first direction to inhibit movement of the dispersion medium toward a second direction perpendicular to the first direction.

18. The microwell device according to claim 17,
wherein the wiping member includes a second surface configured to be brought into abutment with a sealing medium for sealing the microwells to move the sealing medium, and the wiping member includes one or more second partitions configured to extend from the second surface in the first direction to inhibit movement of the sealing medium toward the second direction, and
wherein the second partitions are arranged at corresponding positions as those of the first partitions in the second direction.

19. The microwell device according to claim 12, wherein the base plate is formed of a material having translucency.

20. A target substance detection method comprising:
a reaction step of causing the detection particles that each specifically capture a target substance and the target substance to react with each other;
a particle-encapsulating step of performing the particle encapsulation method of claim 1 with the detection particles after the reaction step; and
a detecting step of detecting the detection particles that have each captured the target substance out of the detection particles stored in the respective microwells after the particle-encapsulating step.
